(19) United States
(12) Patent Application Publication
(10) Pub. No.: US 2015/0272928 A1
(43) Pub. Date: Oct. 1, 2015

(54) INDOLE DERIVATIVES

(71) Applicant: UNIVERSITY OF CENTRAL LANCASHIRE, Preston, Lancashire (GB)

(72) Inventor: Timothy J. Snape, Preston (GB)

(73) Assignee: University of Central Lancashire, Preston (GB)

(21) Appl. No.: 14/431,574

(22) PCT Filed: Sep. 26, 2013
(86) PCT No.: PCT/GB2013/052519

§ 371 Date: Mar. 26, 2015

(30) Foreign Application Priority Data
Sep. 27, 2012 (GB) 1217285.4

(51) Int. Cl. A61K 31/404; A61K 45/06

(52) U.S. Cl. CPC A61K 31/404; A61K 45/06

(57) ABSTRACT

The present invention relates to compounds for use as therapeutic agents, particularly in the treatment and/or prevention of proliferative disorders, such as cancer, especially brain cancers/tumours, wherein the compounds are generally defined by the formula I: wherein A is an aryl or heteroaryl ring system; and R₁ to R₄ are various possible substituent groups, provided that at least one of an R₂ group, the R₃ group, or an R₄ group is or comprises a hydroxy, amino, NH(Rₓ), or mercapto group.

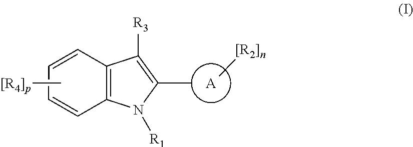

### FIELD OF THE INVENTION

The present invention relates to compounds for use as therapeutic agents, particularly in the treatment and/or prevention of proliferative disorders, such as cancer, especially brain cancers/tumours. The present invention also relates to pharmaceutical compositions comprising said compounds, and to certain specific therapeutic uses of said compounds.

### BACKGROUND OF THE INVENTION

Proliferative disorders, such as cancer, are caused by uncontrolled and unregulated cellular proliferation. Wide ranging research has over the years lead to the development of numerous anticancer drug treatments, though it is becoming increasingly recognised that particular types of cancers need to be targeted with particular drugs. As such, chemotherapy research is increasingly focussing on specific treatments for specific types of cancer rather than generic cancer treatments.

The discovery of alternative and efficacious drug treatments for brain cancers and brain tumours has proved particularly challenging for researchers. For instance, to be effective, such drugs must be capable of penetrating the blood-brain barrier, and be efficacious whilst minimising undesirable side-effects.

Brain cancer drugs currently in use include inter alia Cisplatin, Temozolomide, Etoposide, and Carmustine. Amongst these particular drugs, Cisplatin has been shown to possess the best cytotoxic effects against established glioma cell lines 1321N1 (WHO Grade 2) and U87MG (WHO Grade 4).[¹] It is thought that Cisplatin derives its therapeutic activity against brain cancers from DNA-alkylation behaviour.^{[2]}

It is therefore an object of the present invention to provide an alternative therapeutic treatment of proliferative disorders such as brain cancers.

Another object of the present invention is to provide a drug which operates, at least in part, via a mode of action dissimilar to drugs of the prior art, such as Cisplatin.

Another object of the present invention is to provide a drug which is suitable for treating proliferative disorders, such as brain cancers, and is straightforward to synthesize.

### SUMMARY OF THE INVENTION

According to a first aspect of the present invention, there is provided a compound, or a pharmaceutically acceptable salt, hydrate or solvate thereof, as defined herein.

According to a second aspect of the present invention, there is provided a pharmaceutical composition comprising a compound, or a pharmaceutically acceptable salt, hydrate or solvate thereof, as defined herein in admixture with a pharmaceutically acceptable diluent or carrier.

According to a third aspect of the present invention, there is provided a method of inhibiting growth of either or both U87MG and 1321N1 cell lines in vitro or in vivo, said method comprising contacting a cell with an effective amount of a compound, or a pharmaceutically acceptable salt, hydrate or solvate thereof, as defined herein.

According to a fourth aspect of the present invention, there is provided a method of inhibiting cell proliferation, in vitro or in vivo, said method comprising contacting a cell with an effective amount of a a compound, or a pharmaceutically acceptable salt, hydrate or solvate thereof, as defined herein.

According to a fifth aspect of the present invention, there is provided a method of treating a proliferative disorder in a patient in need of such treatment, said method comprising administering to said patient a therapeutically effective amount of a compound, or a pharmaceutically acceptable salt, hydrate or solvate thereof, or a pharmaceutical composition as defined herein.

According to a sixth aspect of the present invention, there is provided a method of treating cancer in a patient in need of such treatment, said method comprising administering to said patient a therapeutically effective amount of a compound, or a pharmaceutically acceptable salt, hydrate or solvate thereof, or a pharmaceutical composition as defined herein.

According to a seventh aspect of the present invention, there is provided a compound, or a pharmaceutically acceptable salt, hydrate or solvate thereof, or a pharmaceutical composition as defined herein for use in therapy.

According to an eighth aspect of the present invention, there is provided a compound, or a pharmaceutically acceptable salt, hydrate or solvate thereof, or a pharmaceutical composition as defined herein for use in the treatment of a proliferative condition.

According to a ninth aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. In a particular embodiment, the cancer is brain cancer.

According to a tenth aspect of the present invention, there is provided a compound, or a pharmaceutically acceptable salt, hydrate or solvate thereof, as defined herein for use in the inhibition of growth of either or both U87MG and 1321N1 cell lines.

According to an eleventh aspect of the present invention, there is provided a use of a compound, or a pharmaceutically acceptable salt, hydrate or solvate thereof, as defined herein in the manufacture of a medicament for the treatment of a proliferative condition.

According to a twelfth aspect of the present invention, there is provide a use of a compound, or a pharmaceutically acceptable salt, hydrate or solvate thereof, as defined herein in the manufacture of a medicament for the treatment of cancer.

According to a thirteenth aspect of the present invention, there is provided a use of a compound, or a pharmaceutically acceptable salt, hydrate or solvate thereof, as defined herein in the manufacture of a medicament for use in the inhibition of growth of either or both U87MG and 1321N1 cell lines.

According to a fourteenth aspect of the present invention, there is provided a process for preparing a compound, or a pharmaceutically acceptable salt, hydrate or solvate thereof, as defined herein.

According to a fifteenth aspect of the present invention, there is provided a compound, or a pharmaceutically acceptable salt, hydrate or solvate thereof, obtainable by, or obtained by, or directly obtained by a process of preparing a compound as defined herein.

According to a sixteenth aspect of the present invention, there are provided novel intermediates as defined herein which are suitable for use in any one of the synthetic methods set out herein.

Suitably, the proliferative disorder is cancer, suitably a human cancer, suitably brain cancer (and/or associated tumours).

Features, including optional, suitable, and preferred features in relation to one aspect of the invention may also be features, including optional, suitable and preferred features in relation to any other aspect of the invention.

### BRIEF DESCRIPTION OF THE DRAWINGS

Embodiments of the invention are further described hereinafter with reference to the accompanying drawings, in which:

FIG. 1 shows H₂DCFDA staining intensity as quantified by flow cytometry. 1321N1 cells were treated with analogues of compound 4 for 1 h. The various charts of FIG. 1 show (A) Cells+Dye. (B) Cells treated with positive control [100 μM tert-butyl hydroperoxide] for 1 h. (C) Cells treated with indole 2 (500 μM) for 1 h. (D) Cells treated with indole 3 (500 μM) for 1 h. (E) Cells treated with indole 4 (500 μM) for 1 h. (F) Cells treated with indole 7 (500 μM) for 1 h. (Right) U87MG cells were treated with analogues of compound 4 for 1 h. (A) Cells+Dye. (B) Cells treated with positive control [100 μM tert-butyl hydroperoxide (TBHP)] for 1 h. (C) Cells treated with indole 2 (500 μM) for 1 h. (D) Cells treated with indole 3 (500 μM) for 1 h. (E) Cells treated with indole 4 (500 μM) for 1 h. (F) Cells treated with indole 7 (500 μM) for 1 h.

FIG. 2 shows acridine orange staining intensity, as measured by flow cytometry, of acidic vesicular organelle (AVO) formation in 1321N1 cells (Left) and U87MG cells (Right). The various charts of FIG. 2 show (A) Cells only. (B) Cells+acridine orange. (C) Cells treated with indole 4 (500 μM) for 1 h+acridine orange.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring.

Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

The indole positions for the compound of formula I are numbered as follows:

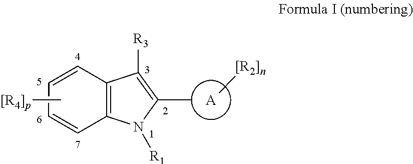

### Compounds of the Invention

The present invention provides a compound of Formula I:

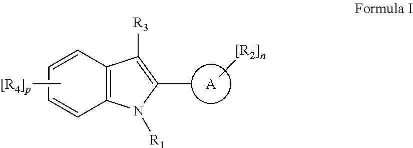

wherein:

R₁ is selected from the group including hydrogen, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulphamoyl, N,N-di-[(1-6C)alkyl]sulphamoyl, or from a group of the formula:

-L^{1a}-X^{1a}
wherein:
   L^{1a} is absent or is selected from SO, SO₂, CO, C(O)O, CH(OR₁ₐ), CON(R₁ₐ), SO₂N(R₁ₐ), wherein R₁ₐ is hydrogen or (1-8C)alkyl; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₁ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{1b}-X^{1b}

wherein:
L^{1b} is absent or is selected from O, S, SO, SO₂, N(R_{1b}), CO, C(O)O, CH(OR_{1b}), CON(R_{1b}), N(R_{1b})CO, N(R₁ₐ)CON(R_{1b}), SO₂N(R_{1b}), N(R_{1b})SO₂, OC(R_{1b})₂, SC(R_{1b})₂ and N(R_{1b})C(R_{1b})₂, wherein R_{1b} is hydrogen or (1-8C)alkyl; and
   X^{1b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

A is an aryl or heteroaryl ring system;

n is 0, 1, 2, 3, or 4;

each R₂ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2a}-X^{2a}
wherein:
   L^{2a} is absent or is selected from O, S, SO, SO₂, N(R₂ₐ), CO, C(O)O, CH(OR₂ₐ), CON(R₂ₐ), N(R₂ₐ)CO, N(R₂ₐ)CON(R₂ₐ), SO₂N(R₂ₐ), N(R₂ₐ)SO₂, OC(R₂ₐ)₂, SC(R₂ₐ)₂ and N(R₂ₐ)C(R₂ₐ)₂, wherein R₂ₐ is hydrogen or (1-8C)alkyl; and
   X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R₂ group is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2b}-X^{2b}

wherein:
L^{2b} is absent or is selected from O, S, SO, SO₂, N(R_{2b}), CO, C(O)O, CH(OR_{2b}), CON(R_{2b}), N(R_{2b})CO, N(R₂ₐ)CON(R_{2b}), SO₂N(R_{2b}), N(R_{2b})SO₂, OC(R_{2b})₂, SC(R_{2b})₂ and N(R_{2b})C(R_{2b})₂, wherein R_{2b} is hydrogen or (1-8C)alkyl; and
   X^{2b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

R₃ is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{3a}-X^{3a}
wherein:
   L^{3a} is absent or is selected from O, S, SO, SO₂, N(R₃ₐ), CO, C(O)O, CH(OR₃ₐ), CON(R₃ₐ), N(R₃ₐ)CO, N(R₃ₐ)CON(R₃ₐ), SO₂N(R₃ₐ), N(R₃ₐ)SO₂, OC(R₃ₐ)₂, SC(R₃ₐ)₂ and N(R₃ₐ)C(R₃ₐ)₂, wherein R₃ₐ is hydrogen or (1-8C)alkyl; and
   X^{3a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₃ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{3b}-X^{3b}

wherein:
L^{3b} is absent or is selected from O, S, SO, SO₂, N(R_{3b}), CO, C(O)O, CH(OR_{3b}), CON(R_{3b}), N(R_{3b})CO, N(R₃ₐ)CON(R_{3b}), SO₂N(R_{3b}), N(R_{3b})SO₂, OC(R_{3b})₂, SC(R_{3b})₂ and N(R_{3b})C(R_{3b})₂, wherein R_{3b} is hydrogen or (1-8C)alkyl; and
   X^{3b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

p is 0, 1, 2, 3, or 4;

each R₄ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N, N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{4a}-X^{4a}
wherein:
   L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-8C)alkyl; and
   X^{4a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₄ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{4b}-X^{4b}
wherein:
   L^{4b} is absent or is selected from O, S, SO, SO₂, N(R_{4b}), CO, C(O)O, CH(OR_{4b}), CON(R_{4b}), N(R_{4b})CO, N(R₄ₐ)CON(R_{4b}), SO₂N(R_{4b}), N(R_{4b})SO₂, OC(R_{4b})₂, SC(R_{4b})₂ and N(R_{4b})C(R_{4b})₂, wherein R_{4b} is hydrogen or (1-8C)alkyl; and
      X^{4b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

wherein at least one of an R₂ group, the R₃ group, or an R₄ group is or comprises a hydroxy, amino, NH(Rₓ), or mercapto group, wherein Rₓ is any acceptable group, defined herein in relation to R₂, R₃, and R₄, for attachment to a nitrogen atom (especially an NH group); or a pharmaceutically acceptable salt, hydrate or solvate thereof.

The present invention also provides a compound for use in the treatment of a brain cancer and/or brain tumour, the compound defined by Formula Ib:

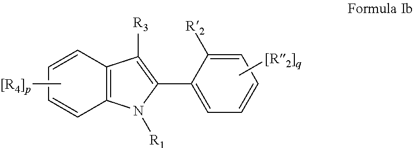

wherein:

R₁ is selected from the group including hydrogen, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulphamoyl, N,N-di-[(1-6C)alkyl]sulphamoyl, or from a group of the formula:

-L^{1a}-X^{1a}
wherein:
   L^{1 a} is absent or is selected from SO, SO₂, CO, C(O)O, CH(OR₁ₐ), CON(R₁ₐ), SO₂N(R₁ₐ), wherein R₁ₐ is hydrogen or (1-8C)alkyl; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₁ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{1b}-X^{1b}

wherein:
L^{1b} is absent or is selected from O, S, SO, SO₂, N(R_{1b}), CO, C(O)O, CH(OR_{1b}), CON(R_{1b}), N(R_{1b})CO, N(R₁ₐ)CON(R_{1b}), SO₂N(R_{1b}), N(R_{1b})SO₂, OC(R_{1b})₂, SC(R_{1b})₂ and N(R_{1b})C(R_{1b})₂, wherein R_{1b} is hydrogen or (1-8C)alkyl; and
   X^{1b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

R′₂ is hydrogen or is selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2a}-X^{2a}
wherein:
   L^{2a} is absent or is selected from O, S, SO, SO₂, N(R₂ₐ), CO, C(O)O, CH(OR₂ₐ), CON(R₂ₐ), N(R₂ₐ)CO, N(R₂ₐ)CON(R₂ₐ), SO₂N(R₂ₐ), N(R₂ₐ)SO₂, OC(R₂ₐ)₂, SC(R₂ₐ)₂ and N(R₂ₐ)C(R₂ₐ)₂, wherein R₂ₐ is hydrogen or (1-8C)alkyl; and X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R′₂ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

L^{2b}-X^{2b}

wherein:
L^{2b} is absent or is selected from O, S, SO, SO₂, N(R_{2b}), CO, C(O)O, CH(OR_{2b}), CON(R_{2b}), N(R_{2b})CO, N(R₂ₐ)CON(R_{2b}), SO₂N(R_{2b}), N(R_{2b})SO₂, OC(R_{2b})₂, SC(R_{2b})₂ and N(R_{2b})C(R_{2b})₂, wherein R_{2b} is hydrogen or (1-8C)alkyl; and
   X^{2b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

q is 0, 1, 2, or 3;

each R″₂ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2a}-X^{2a};
wherein each R″₂ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2b}-X^{2b};

R₃ is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{3a}-X^{3a}
wherein:
   L^{3a} is absent or is selected from O, S, SO, SO₂, N(R₃ₐ), CO, C(O)O, CH(OR₃ₐ), CON(R₃ₐ), N(R₃ₐ)CO, N(R₃ₐ)CON(R₃ₐ), SO₂N(R₃ₐ), N(R₃ₐ)SO₂, OC(R₃ₐ)₂,

SC(R₃ₐ)₂ and N(R₃ₐ)C(R₃ₐ)₂, wherein R₃ₐ is hydrogen or (1-8C)alkyl; and X^{3a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₃ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{3b}-X^{3b}

wherein:
L^{3b} is absent or is selected from O, S, SO, SO₂, N(R_{3b}), CO, C(O)O, CH(OR_{3b}), CON(R_{3b}), N(R_{3b})CO, N(R₃ₐ)CON(R_{3b}), SO₂N(R_{3b}), N(R_{3b})SO₂, OC(R_{3b})₂, SC(R_{3b})₂ and N(R_{3b})C(R_{3b})₂, wherein R_{3b} is hydrogen or (1-8C)alkyl; and
   X^{3b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

p is 0, 1, 2, 3, or 4;

each R₄ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{4a}-X^{4a}
wherein:
   L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-8C)alkyl; and
   X^{4a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₄ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{4b}-X^{4b}

wherein:
L^{4b} is absent or is selected from O, S, SO, SO₂, N(R_{4b}), CO, C(O)O, CH(OR_{4b}), CON(R_{4b}), N(R_{4b})CO, N(R₄ₐ)CON(R_{4b}), SO₂N(R_{4b}), N(R_{4b})SO₂, OC(R_{4b})₂, SC(R_{4b})₂ and N(R_{4b})C(R_{4b})₂, wherein R_{4b} is hydrogen or (1-8C)alkyl; and
   X^{4b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

wherein at least one of the R′₂ group, the R₃ group, or an R₄ group in the 4-indole position is or comprises a hydroxy, amino, NH(Rₓ), or mercapto group, wherein Rₓ is any acceptable group, defined in relation to R′₂, R₃, and R₄, for attachment to a nitrogen atom; or a pharmaceutically acceptable salt, hydrate or solvate thereof.

The present invention also provides a compound for use in the treatment of a brain cancer and/or brain tumour, the compound defined by Formula Id:

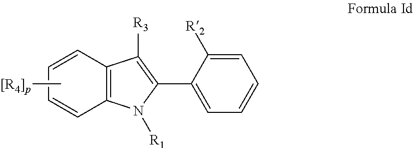

wherein:

R₁ is selected from the group including hydrogen, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulphamoyl, N,N-di-[(1-6C)alkyl]sulphamoyl, or from a group of the formula:

-L^{1a}-X^{1a}
wherein:
   L^{1a} is absent or is selected from SO, SO₂, CO, C(O)O, CH(OR₁ₐ), CON(R₁ₐ), SO₂N(R₁ₐ), wherein R₁ₐ is hydrogen or (1-8C)alkyl; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₁ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{1b}-X^{1b}

wherein:
L^{1b} is absent or is selected from O, S, SO, SO₂, N(R_{1b}), CO, C(O)O, CH(OR_{1b}), CON(R_{1b}), N(R_{1b})CO, N(R₁ₐ)CON(R_{1b}), SO₂N(R_{1b}), N(R_{1b})SO₂, OC(R_{1b})₂, SC(R_{1b})₂ and N(R_{1b})C(R_{1b})₂, wherein R_{1b} is hydrogen or (1-8C)alkyl; and
   X^{1b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

R′₂ is hydrogen or is selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2a}-X^{2a}
wherein:
   L^{2a} is absent or is selected from O, S, SO, SO₂, N(R₂ₐ), CO, C(O)O, CH(OR₂ₐ), CON(R₂ₐ), N(R₂ₐ)CO, N(R₂ₐ)CON(R₂ₐ), SO₂N(R₂ₐ), N(R₂ₐ)SO₂, OC(R₂ₐ)₂, SC(R₂ₐ)₂ and N(R₂ₐ)C(R₂ₐ)₂, wherein R₂ₐ is hydrogen or (1-8C)alkyl; and
   X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R′₂ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{2b}-X^{2b}

wherein:
L^{2b} is absent or is selected from O, S, SO, SO₂, N(R_{2b}), CO, C(O)O, CH(OR_{2b}), CON(R_{2b}), N(R_{2b})CO, N(R₂ₐ)CON(R_{2b}), SO₂N(R_{2b}), N(R_{2b})SO₂, OC(R_{2b})₂, SC(R_{2b})₂ and N(R_{2b})C(R_{2b})₂, wherein R_{2b} is hydrogen or (1-8C)alkyl; and
   X^{2b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

R₃ is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{3a}-X^{3a}
wherein:
   L^{3a} is absent or is selected from O, S, SO, SO₂, N(R₃ₐ), CO, C(O)O, CH(OR₃ₐ), CON(R₃ₐ), N(R₃ₐ)CO, N(R₃ₐ)CON(R₃ₐ), SO₂N(R₃ₐ), N(R₃ₐ)SO₂, OC(R₃ₐ)₂, SC(R₃ₐ)₂ and N(R₃ₐ)C(R₃ₐ)₂, wherein R₃ₐ is hydrogen or (1-8C)alkyl; and
   X^{3a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₃ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{3b}-X^{3b}

wherein:
L^{3b} is absent or is selected from O, S, SO, SO₂, N(R_{3b}), CO, C(O)O, CH(OR_{3b}), CON(R_{3b}), N(R_{3b})CO, N(R₃ₐ)CON(R_{3b}), SO₂N(R_{3b}), N(R_{3b})SO₂, OC(R_{3b})₂, SC(R_{3b})₂ and N(R_{3b})C(R_{3b})₂, wherein R_{3b} is hydrogen or (1-8C)alkyl; and
   X^{3b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

p is 0, 1, 2, 3, or 4;

each R₄ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-L^{4a}-X^{4a}
wherein:
   L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-8C)alkyl; and
   X^{4a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₄ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:

-L^{4b}-X^{4b}

wherein:
L^{4b} is absent or is selected from O, S, SO, SO₂, N(R_{4b}), CO, C(O)O, CH(OR_{4b}), CON(R_{4b}), N(R_{4b})CO, N(R₄ₐ)CON(R_{4b}), SO₂N(R_{4b}), N(R_{4b})SO₂, OC(R_{4b})₂, SC(R_{4b})₂ and N(R_{4b})C(R_{4b})₂, wherein R_{4b} is hydrogen or (1-8C)alkyl; and
   X^{4b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;

wherein at least one of an R₂ group, the R₃ group, or an R₄ group is or comprises a hydroxy, amino, NH(Rₓ), or mercapto group, wherein Rₓ is any acceptable group, defined in relation to R′₂, R₃, and R₄, for attachment to a nitrogen atom (especially an NH group); or a pharmaceutically acceptable salt, hydrate or solvate thereof.

As the person skilled in the art will readily appreciate, any R₂ definitions presented herein may suitably be definitions for R′₂ and/or suitably definitions for R″₂.

The indole positions for the compound of formula I are numbered as follows:

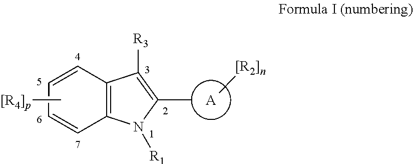

Particular compounds of the invention include, for example, compounds of the formula I, or pharmaceutically acceptable salts thereof, wherein, unless otherwise stated, each of Ring System A, n, p, R₁, L^{1a}, L^{1b}, X^{1a}, X^{1b}, R₁ₐ, R_{1b}, R₂, L^{2a}, L^{2b}, X^{2a}, X^{2b}, R₂ₐ, R_{2b}, R₃, L^{3a}, L^{3b}, X^{3a}, X^{3b}, R₃ₐ, R_{3b}, R₄, L^{4a}, L^{4b}, X^{4a}, X^{4b}, R₄ₐ, R_{4b}, and Rₓ has any of the meanings defined hereinbefore or in any of paragraphs (1) to (74) hereinafter:—

(1) R₁ is selected from the group including hydrogen, carboxy, (1-8C)alkyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulphamoyl, N,N-di-[(1-6C)alkyl]sulphamoyl, or from a group of the formula:

-L^{1a}-X^{1a}
wherein:
   L^{1 a} is absent or is selected from SO, SO₂, CO, C(O)O; and
   X^{1a} is aryl, aryl-(1-6C)alkyl;
wherein R₁ is optionally further substituted as defined herein.
   (2) R₁ is selected from the group including hydrogen, carboxy, (1-4C)alkyl, (1-4C)hydroxyalkyl, (1-4C)alkylsulphinyl, (1-4C)alkylsulphonyl, (1-4C)alkoxycarbonyl, N-(1-4C)alkylcarbamoyl, N,N-di-[(1-4C)alkyl]carbamoyl, (2-4C)alkanoyl, N-(1-4C)alkylsulphamoyl, N,N-di-[(1-4C)alkyl]sulphamoyl, or from a group of the formula:

L^{1a}X^{1a}
wherein:
   L^{1a} is absent or is selected from CO, C(O)O; and
   X^{1a} is aryl, aryl-(1-2C)alkyl
wherein R₁ is optionally further substituted as defined herein.
   (3) R₁ is selected from the group including hydrogen, carboxy, (1-4C)alkyl, (2-4C)alkanoyl, or from a group of the formula:

-L^{1a}-X^{1a}
wherein:
   L^{1a} is absent or is selected from CO, C(O)O; and
   X^{1a} is phenyl or benzyl;
wherein R₁ is optionally further substituted as defined herein.
   (4) optional further substituents for R₁ are selected from one or more halogeno, (1-4C)alkyl, hydroxy, mercapto, amino.
   (5) R₁ is hydrogen, acetate, benzoate, (1-4C)alkyl, or benzyl.
   (6) R₁ is hydrogen.
   (7) A is an aryl or heteroaryl ring system defined by:

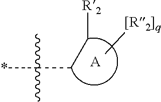

wherein * indicates the indole carbon to which Ring System A is attached, R′₂ is hydrogen or has any of the meanings defined herein in relation to an R₂ group, R″₂ has any of the meanings defined herein in relation to an R₂ group, and q is 0, 1, 2, or 3.
   (8) A is a monocyclic aryl or heteroaryl ring system;
   (9) A is a 5- or 6-membered aryl or heteroaryl ring system.
   (10) A is a 6-membered aryl or heteroaryl ring system.
   (11) A is an aryl ring system as defined herein.
   (12) A is a phenyl ring system (i.e. benzene).
   (13) n is 0, 1, or 2.
   (14) n is 0 or 1.
   (15) n is 1.
   (16) q is n−1 (i.e. R′₂ is present as the first R₂ group).
   (17) q is 0 or 1.
   (18) q is 0.
   (19) each R₂ group, which may be the same or different, is independently selected from halogeno, trifluoromethyl, cyano, nitro, hydroxy, mercapto, amino, formyl, carboxy, carbamoyl, ureido, (1-4C)alkyl, (1-4C)hydroxyalkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-5C)alkanoyl, (2-5C)alkanoyloxy, (2-5C)alkanoylamino, N-(1-4C)alkyl-(2-5C)alkanoylamino, N,N-di-[(1-4C)alkyl]sulphamoyl, (1-4C)alkanesulphonylamino and N-(1-4C)alkyl-(1-4C)alkanesulphonylamino, or from a group of the formula:

-L^{2a}-X^{2a}
wherein:
   L^{2a} is absent or is selected from O, S, SO, SO₂, N(R₂ₐ), CO, C(O)O, CH(OR₂ₐ), CON(R₂ₐ), N(R₂ₐ)CO, N(R₂ₐ)CON(R₂ₐ), SO₂N(R₂ₐ), N(R₂ₐ)SO₂, OC(R₂ₐ)₂, SC(R₂ₐ)₂ and N(R₂ₐ)C(R₂ₐ)₂, wherein R₂ₐ is hydrogen or (1-2C)alkyl; and
   X^{2a} is aryl, aryl-(1-2C)alkyl, heteroaryl, heteroaryl-(1-2C)alkyl, heterocyclyl or heterocyclyl-(1-2C)alkyl;
wherein any R₂ group is optionally further substituted as defined herein.
   (20) each R₂ group, which may be the same or different, is independently selected from halogeno, trifluoromethyl, cyano, nitro, hydroxy, mercapto, amino, formyl, carboxy, carbamoyl, (1-4C)alkylsulphinyl, (1-4C)alkylsulphonyl, (1-4C)alkylamino, (1-4C)alkoxycarbonyl, N-(1-4C)alkylcarbamoyl, N,N-di-[(1-4C)alkyl]carbamoyl, (2-5C)alkanoyl, (2-5C)alkanoylamino, N-(1-4C)alkyl-(2-5C)alkanoylamino, N,N-di-[(1-4C)alkyl]sulphamoyl, (1-4C)alkanesulphonylamino and N-(1-4C)alkyl-(1-4C)alkanesulphonylamino, or from a group of the formula:

-L^{2a}-X^{2a}
wherein:
   L^{2a} is selected from SO, SO₂, N(R₂ₐ), CO, C(O)O, CON(R₂ₐ), N(R₂ₐ)CO, SO₂N(R₂ₐ), and N(R₂ₐ)SO₂, wherein R₂ₐ is hydrogen or (1-2C)alkyl; and
   X^{2a} is aryl, aryl-(1-2C)alkyl, heteroaryl, heteroaryl-(1-2C)alkyl, heterocyclyl or heterocyclyl-(1-2C)alkyl;
wherein any R₂ group is optionally further substituted as defined herein.
   (21) each R₂ group, which may be the same or different, is independently selected from cyano, nitro, hydroxy, formyl, carboxy, (2-5C)alkanoyl;
wherein any R₂ group is optionally further substituted as defined herein.
   (22) each R₂ group, which may be the same or different, is independently selected from hydroxy, mercapto, amino, ureido, (1-4C)alkyl, (1-4C)hydroxyalkyl, (1-4C)alkoxy, (1-4C)alkylthio, (1-4C)alkylamino, di-[(1-4C)alkyl]amino, (2-5C)alkanoyloxy, (2-5C)alkanoylamino, N-(1-4C)alkyl-(2-5C)alkanoylamino, (1-4C)alkanesulphonylamino and N-(1-4C)alkyl-(1-4C)alkanesulphonylamino, or from a group of the formula:

-L^{2a}-X^{2a}
wherein:
   L^{2a} is absent or is selected from O, S, N(R₂ₐ), CH(OR₂ₐ), N(R₂ₐ)CO, N(R₂ₐ)CON(R₂ₐ), N(R₂ₐ)SO₂, OC(R₂ₐ)₂, SC(R₂ₐ)₂ and N(R₂ₐ)C(R₂ₐ)₂, wherein R₂ₐ is hydrogen or (1-2C)alkyl; and
   X^{2a} is aryl, aryl-(1-2C)alkyl, heteroaryl, heteroaryl-(1-2C)alkyl, heterocyclyl or heterocyclyl-(1-2C)alkyl;
wherein any R₂ group is optionally further substituted as defined herein.
   (23) each R₂ group, which may be the same or different, is independently selected from hydroxy, mercapto, amino, ureido, (1-4C)alkyl, (1-4C)hydroxyalkyl, (1-4C)alkoxy, (1-4C)alkylthio, (1-4C)alkylamino, di-[(1-4C)alkyl]amino,
wherein any R₂ group is optionally further substituted as defined herein.
   (24) at least one R₂ group is or comprises a group selected from hydroxy, amino, NH(Rₓ), or mercapto, wherein Rₓ is any acceptable, defined herein in relation to R₂, for attachment to a nitrogen atom (especially an NH group).
   (25) at least one R₂ group is or comprises a hydroxy.
   (26) at least one R₂ group is hydroxy.
   (27) at most one R₂ group is or comprises a group selected from hydroxy, amino, NH(Rₓ), or mercapto, wherein Rₓ is any acceptable group, defined herein in relation to R₂, for attachment to a nitrogen atom (especially an NH group).
   (28) at most one R₂ group is hydroxy.
   (29) R′₂ is hydrogen or R′₂ is or comprises a group selected from hydroxy, amino, NH(Rₓ), or mercapto, wherein Rₓ is any acceptable group, defined herein in relation to R₂, for attachment to a nitrogen atom (especially an NH group).
   (30) R′₂ is hydrogen or R′₂ is a group selected from hydroxy, amino, NH(Rₓ), or mercapto, wherein Rₓ is any acceptable group, defined herein in relation to R₂, for attachment to a nitrogen atom (especially an NH group).
   (31) R′₂ is hydrogen or hydroxy.
   (32) R′₂ is hydroxy.
   (33) Each R″₂ group is an electron withdrawing group.
   (34) Each R″₂ group is an electron donating group.
   (35) Each R″₂ group is hydroxy.
   (36) optional further substituents for R₂ are selected from one or more halogeno, (1-4C)alkyl, hydroxy, mercapto, amino.
   (37) R₃ is selected from hydrogen, trifluoromethyl, hydroxy, mercapto, amino, formyl, carboxy, carbamoyl, (1-4C)alkyl, (2-4C)alkenyl, (2-4C)alkynyl, (1-4C)hydroxyalkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, N-(1-4C)alkyl-(2-4C)alkanoylamino, N,N′,N-tri-[(1-4C)alkyl]ureido, N-(1-4C)alkylsulphamoyl, N,N-di-[(1-4C)alkyl]sulphamoyl, (1-4C)alkanesulphonylamino and N-(1-4C)alkyl-(1-4C)alkanesulphonylamino, or from a group of the formula:

-L^{3a}-X^{3a}
wherein:
   L^{3a} is absent or is selected from O, S, SO, SO₂, N(R₃ₐ), CO, C(O)O, CH(OR₃ₐ), CON(R₃ₐ), N(R₃ₐ)CO, SO₂N(R₃ₐ), N(R₃ₐ)SO₂, OC(R₃ₐ)₂, SC(R₃ₐ)₂ and N(R₃ₐ)C(R₃ₐ)₂, wherein R₃ₐ is hydrogen or (1-8C)alkyl; and
   X^{3a} is aryl, aryl-(1-2C)alkyl, (5-6C)cycloalkyl, (5-6C)cycloalkyl-(1-4C)alkyl, (5-6C)cycloalkenyl, (5-6C)cycloalkenyl-(1-4C)alkyl, heteroaryl, heteroaryl-(1-4C)alkyl, heterocyclyl or heterocyclyl-(1-4C)alkyl;
wherein R₃ is optionally further substituted as described herein.
   (38) R₃ is hydrogen or is or comprises a group selected from hydroxy, amino, NH(Rₓ), or mercapto, wherein Rₓ is any acceptable group, defined herein in relation to R₃, for attachment to a nitrogen atom (especially an NH group).
   (39) R₃ is or comprises a hydroxy.
   (40) R₃ is selected from hydrogen, hydroxy, (1-4C)hydroxyalkyl, wherein R₃ is optionally further substituted as described herein.
   (41) R₃ is selected from hydrogen, and (1-2C)hydroxyalkyl.
   (42) R₃ is hydrogen or hydroxymethyl.
   (43) R₃ is hydroxymethyl.
   (44) R₃ is hydroxy.
   (45) optional further substituents for R₃ are selected from one or more halogeno, (1-4C)alkyl, hydroxy, mercapto, amino.
   (46) p is 0, 1, or 2;
   (47) p is 0.
   (48) each R₄ group, which may be the same or different, is independently selected from halogeno, trifluoromethyl, cyano, isocyano, nitro, hydroxy, mercapto, amino, formyl, carboxy, carbamoyl, (1-4C)alkyl, (1-4C)hydroxyalkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, N-(1-4C)alkyl-(2-4C)alkanoylamino, N,N-di-[(1-4C)alkyl]sulphamoyl, (1-4C)alkanesulphonylamino and N-(1-4C)alkyl-(1-4C)alkanesulphonylamino, or from a group of the formula:

-L^{4a}-X^{4a}
wherein:
   L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-2C)alkyl; and
   X^{4a} is aryl, aryl-(1-2C)alkyl, (5-6C)cycloalkyl, (5-6C)cycloalkyl-(1-2C)alkyl, (5-6C)cycloalkenyl, (5-6C)cycloalkenyl-(1-2C)alkyl, heteroaryl, heteroaryl-(1-2C)alkyl, heterocyclyl or heterocyclyl-(1-2C)alkyl;
wherein R₄ is optionally further substituted as defined herein.
   (49) each R₄ group, which may be the same or different, is independently selected from halogeno, trifluoromethyl, cyano, isocyano, nitro, hydroxy, mercapto, amino, formyl, carboxy, carbamoyl, (1-4C)alkyl, (1-4C)hydroxyalkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, N-(1-4C)alkyl-(2-4C)alkanoylamino, N,N-di-[(1-4C)alkyl]sulphamoyl, (1-4C)alkanesulphonylamino and N-(1-4C)alkyl-(1-4C)alkanesulphonylamino, or from a group of the formula:

-L^{4a}-X^{4a}
wherein:
   L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-2C)alkyl; and
   X^{4a} is aryl, aryl-(1-2C)alkyl, (5-6C)cycloalkyl, (5-6C)cycloalkyl-(1-2C)alkyl, (5-6C)cycloalkenyl, (5-6C)cycloalkenyl-(1-2C)alkyl, heteroaryl, heteroaryl-(1-2C)alkyl, heterocyclyl or heterocyclyl-(1-2C)alkyl;
wherein R₄ is optionally further substituted as defined herein.
   (50) each R₄ group, which may be the same or different, is independently selected from halogeno, trifluoromethyl, cyano, isocyano, nitro, hydroxy, formyl, carboxy, carbamoyl, (1-4C)hydroxyalkyl, (1-4C)alkylsulphinyl, (1-4C)alkylsulphonyl, (1-4C)alkoxycarbonyl, N-(1-4C)alkylcarbamoyl, N,N-di-[(1-4C)alkyl]carbamoyl, (2-4C)alkanoyl, N,N-di-[(1-4C)alkyl]sulphamoyl, or from a group of the formula:

-L^{4a}-X^{4a}
wherein:
   L^{4a} is selected from SO, SO₂, CO, C(O)O, CON(R₄ₐ), SO₂N(R₄ₐ), wherein R₄ₐ is hydrogen or (1-2C)alkyl; and
   X^{4a} is aryl, aryl-(1-2C)alkyl, (5-6C)cycloalkyl, (5-6C)cycloalkyl-(1-2C)alkyl, (5-6C)cycloalkenyl, (5-6C)cycloalkenyl-(1-2C)alkyl, heteroaryl, heteroaryl-(1-2C)alkyl, heterocyclyl or heterocyclyl-(1-2C)alkyl;
wherein R₄ is optionally further substituted as defined herein.
   (51) each R₄ group, which may be the same or different, is independently selected from hydroxy, mercapto, amino, (1-4C)alkyl, (1-4C)hydroxyalkyl, (1-4C)alkoxy, (1-4C)alkylthio, (1-4C)alkylamino, di-[(1-4C)alkyl]amino, (2-4C)alkanoyloxy, (2-4C)alkanoylamino, N-(1-4C)alkyl-(2-4C)alkanoylamino, (1-4C)alkanesulphonylamino and N-(1-4C)alkyl-(1-4C)alkanesulphonylamino, or from a group of the formula:

-L^{4a}-X^{4a}
wherein:
   L^{4a} is absent or is selected from O, S, N(R₄ₐ), CH(OR₄ₐ), N(R₄ₐ)CO, N(R₄ₐ)CON(R₄ₐ), N(R₄ₐ)SO₂, OC(R₄ₐ)₂, SC(R₄ₐ)₂ and N(R₄ₐ)C(R₄ₐ)₂, wherein R₄ₐ is hydrogen or (1-2C)alkyl; and
   X^{4a} is aryl, aryl-(1-2C)alkyl, (5-6C)cycloalkyl, (5-6C)cycloalkyl-(1-2C)alkyl, (5-6C)cycloalkenyl, (5-6C)cycloalkenyl-(1-2C)alkyl, heteroaryl, heteroaryl-(1-2C)alkyl, heterocyclyl or heterocyclyl-(1-2C)alkyl;
wherein R₄ is optionally further substituted as defined herein.
   (52) each R₄ group, which may be the same or different, is independently selected from nitro, hydroxy, mercapto, amino.
   (53) at least one R₄ group is or comprises a group selected from hydroxy, amino, NH(Rₓ), or mercapto, wherein Rₓ is any acceptable group, defined herein in relation to R₄, for attachment to a nitrogen atom (especially an NH group).
   (54) at least one R₄ group is or comprises a hydroxy.
   (55) at least one R₄ group is hydroxy.
   (56) at most one R₄ group is or comprises a group selected from hydroxy, amino, NH(Rₓ), or mercapto, wherein Rₓ is any acceptable group, defined herein in relation to R₄, for attachment to a nitrogen atom (especially an NH group).
   (57) at most one R₄ group is hydroxy.
   (58) optional further substituents for R₄ are selected from one or more halogeno, (1-4C)alkyl, hydroxy, mercapto, amino.
   (59) at least one of an R₂ group or the R₃ group is or comprises a hydroxy, amino, NH(Rₓ), or mercapto group, wherein Rₓ is any acceptable group, defined herein in relation to R₂ and R₃, for attachment to a nitrogen atom (especially an NH group).
   (60) at least one of the R′₂ group, the R₃ group, or an R₄ group in the 4-indole position, is or comprises a hydroxy, amino, NH(Rₓ), or mercapto group, wherein Rₓ is any acceptable group, defined herein in relation to R₂, R₃, and R₄, for attachment to a nitrogen atom (especially an NH group).
   (61) at least one of the R′₂ group and the R₃ group is or comprises a hydroxy, amino, NH(Rₓ), or mercapto group, wherein Rₓ is any acceptable group, defined herein in relation to R₂ and R₃, for attachment to a nitrogen atom (especially an NH group).
   (62) at least one of an R₂ group, the R₃ group, or an R₄ group is or comprises a hydroxy.
   (63) at least one of an R₂ group, the R₃ group, or an R₄ group is hydroxy or hydroxymethyl.
   (64) at least one of an R₂ group and the R₃ group is or comprises a hydroxy.
   (65) at least one of the R′₂ group, the R₃ group, or an R₄ group in the 4-indole position, is or comprises a hydroxy.
   (66) at least one of R′₂ and R₃ is or comprises a hydroxy.
   (67) at least one of R′₂ and R₃ is hydroxy or (1-3C)hydroxyalkyl.
   (68) at least one of R′₂ and R₃ is hydroxy or hydroxymethyl.
   (69) One of R′₂ or R₃ is or comprises a hydroxy, amino, NH(Rₓ), or mercapto group, wherein Rₓ is any acceptable group, defined herein in relation to R₂ and R₃, for attachment to a nitrogen atom (especially an NH group), and the other of R′₂ or R₃ is hydrogen.
   (70) One of R′₂ or R₃ is or comprises a hydroxy, and the other of R′₂ or R₃ is hydrogen.
   (71) One of R′₂ or R₃ is hydroxy or (1-3C)hydroxyalkyl, and the other of R′₂ or R₃ is hydrogen.
   (72) One of R′₂ or R₃ is hydroxy or hydroxymethyl, and the other of R′₂ or R₃ is hydrogen.
   (73) R′₂ is hydroxy and R₃ is hydrogen; or R′₂ is hydrogen and R₃ is hydroxymethyl.
   (74) At least one of an R₂ group, the R₃ group, or an R₄ group is or comprises a group with potential to form a reactive oxygen species (ROS).

In a particular group of compounds of the invention, the compounds have the structural formula Ia (a sub-definition of formula I):

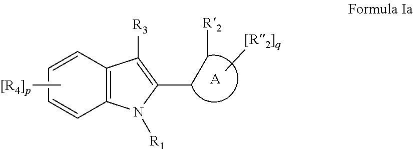

wherein A is an aryl or heteroaryl ring system with an R′₂ group attached to a carbon atom directly adjacent to (i.e. in the ortho-position of A relative to) the carbon atom of Ring System A adjoining the indole, R′₂ is hydrogen or has any of the meanings defined herein in relation to an R₂ group, R″₂ has any of the meanings defined herein in relation to an R₂ group, and q is 0, 1, 2, or 3, and R₁, R₃, R₄, p and any other relevant groups have any one of the meanings defined herein.

In a particular group of compounds of the invention, the compounds have the structural formula Ib (a sub-definition of formula I):

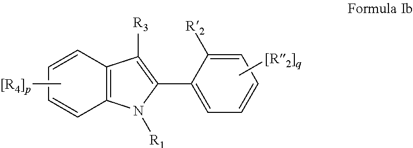

wherein A is a benzene ring system with an R′₂ group ortho- to the benzene carbon atom adjoining the indole, R′₂ is hydrogen or has any of the meanings defined herein in relation to an R₂ group, R″₂ has any of the meanings defined herein in relation to an R₂ group, and q is 0, 1, 2, or 3, and R₁, R₃, R₄, p and any other relevant groups have any one of the meanings defined herein.

In a particular group of compounds of the invention, the compounds have the structural formula Ic (a sub-definition of formula I):

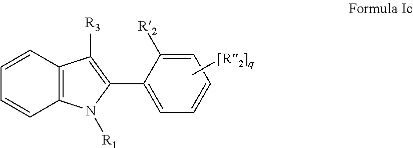

wherein A is a benzene ring system with an R′₂ group ortho- to the benzene carbon atom adjoining the indole, R′₂ is hydrogen or has any of the meanings defined herein in relation to an R₂ group, R″₂ has any of the meanings defined herein in relation to an R₂ group, p is 0, q is 0, 1, 2, or 3, and R₁, R₃, and any other relevant groups have any one of the meanings defined herein.

In a particular group of compounds of the invention, the compounds have the structural formula Id (a sub-definition of formula I):

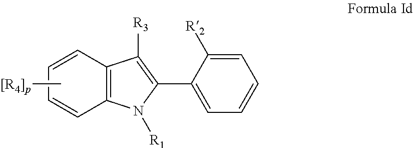

wherein A is a benzene ring system with an R′₂ group ortho- to the benzene carbon atom adjoining the indole, R′₂ is hydrogen or has any of the meanings defined herein in relation to an R₂ group, q is 0, and R₁, R₃, R₄, p and any other relevant groups have any one of the meanings defined herein.

In a particular group of compounds of the invention, the compounds have the structural formula Ie (a sub-definition of formula I):

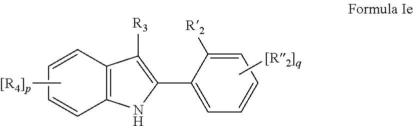

Wherein R₁ is hydrogen, A is a benzene ring system with an R′₂ group ortho- to the benzene carbon atom adjoining the indole, R′₂ is hydrogen or has any of the meanings defined herein in relation to an R₂ group, R″₂ has any of the meanings defined herein in relation to an R₂ group, q is 0, 1, 2, or 3, and R₃, R₄, p and any other relevant groups have any one of the meanings defined herein.

Particular compounds of the invention include those set forth in the Examples, or a pharmaceutically acceptable salt, hydrate or solvate thereof. Particular compounds of the invention include any one of the following:
(2-phenyl-1H-indol-3-yl)methanol;
2-(1H-indol-2-yl)phenol;
   or a pharmaceutically acceptable salt, hydrate or solvate thereof.

The various functional groups and substituents making up the compounds of the formula I are typically chosen such that the molecular weight of the compound of the formula I does not exceed 1000. More usually, the molecular weight of the compound will be less than 750, for example less than 700, or less than 650, or less than 600, or less than 550. More preferably, the molecular weight is less than 525 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶0 and ¹⁸0; and the like.

It is also to be understood that certain compounds of the formula I may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

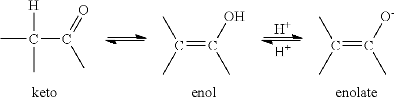

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula I may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I and in-vivo cleavable amide derivatives that may be formed at a carboxy group or an amino group in a compound of the formula I.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups.

Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of Y, R₁, L, Ring A, Ring B and any substituent groups associated therewith. Suitable processes for their preparation are described further in the accompanying Examples.

Once a compound of formula I has been synthesised by any one of the processes defined herein, the processes may then further comprise the additional steps of:

(i) removing any protecting groups present;
(ii) converting the compound formula I into another compound of formula I;
(iii) forming a pharmaceutically acceptable salt, hydrate or solvate thereof; and/or
(iv) forming a prodrug thereof.

An example of (ii) above is when a compound of formula I is synthesised and then one or more of the groups R₁, or Rₐ₋ⱼ may be further reacted to change the nature of the group and provide an alternative compound of formula I. For example, the compound can be reacted to covert R₁ into a substituent group other than hydrogen.

The resultant compounds of formula I can be isolated and purified using techniques well known in the art.

Scheme 1 below depicts a generalised scheme illustrating how the compounds of formula I may be synthesised.

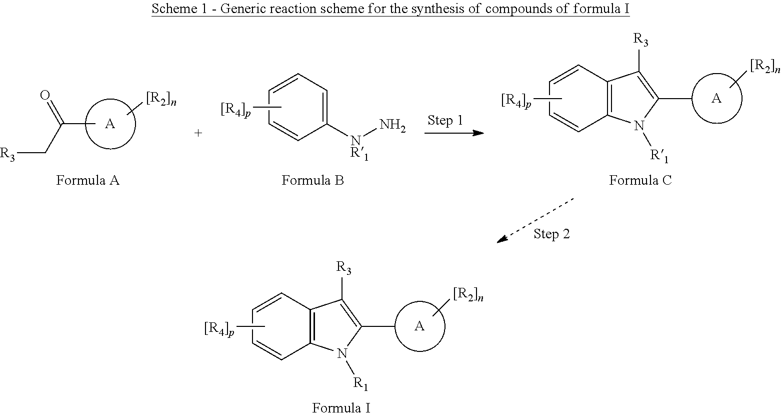

R₁, R₂, R₃, R₄, n, p, Ring System A, and any other associated groups each have any one of the meanings defined herein.

R′₁ may be either R₁, a protected form of R₁, or hydrogen.

Where R′₁ is R₁, there is no need for Step 2. However, in some embodiments, Step 2 involves converting R′₁ to R₁, whether by installing an R₁ group upon the free indole nitrogen (which is NH, i.e. R′₁ is hydrogen), or else modifying the R′₁ group to produce R₁ (e.g. via a deprotection or other suitable transformation).

The compounds of Formula A, B, and C are intermediates from which a variety of compounds of formula I can be made.

Step 1 is typically a two stage process. During the first stage the compounds of Formulae A and B typically condense to yield a corresponding hydrazone intermediate. The hydrazone intermediate may be isolated. During the second stage the hydrazone is transformed, via a rearrangement, to yield the compound of formula C (or formula I directly).

The present invention provides a process for preparing a compound of formula I (as defined herein), the process comprising the steps of:

reacting a compound of formula A:

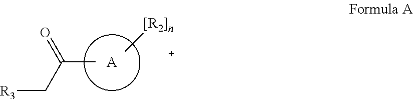

with a compound of formula B:

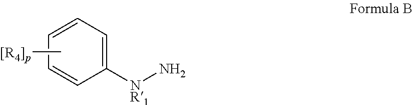

to form a compound of formula C:

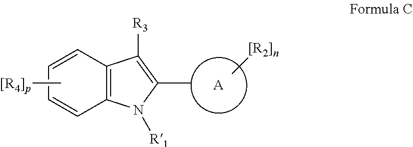

wherein R₁, R₂, R₃, R₄, n, p, Ring System A, and any other associated groups each have any one of the meanings defined herein, and R′₁ is either R₁, a protected form of R₁, or hydrogen; and optionally thereafter (and if necessary—i.e. where R′₁ is not the same as R₁):
(i) Transforming the compound of Formula C into the compound of Formula I by, for example, converting R′₁ to R₁, whether by installing an R₁ group upon the free indole nitrogen (i.e. where R′₁ is hydrogen), or else otherwise modifying the R′₁ group to produce R₁ (e.g. via a deprotection or other suitable transformation);
(ii) removing any protecting groups present into R₁;
(iii) converting the compound formula I into another compound of formula I;
(iv) forming a pharmaceutically acceptable salt, hydrate or solvate thereof; and/or
(v) forming a prodrug thereof.

As suggested above, the reaction of compounds of formulae A and B to produce a compound of formula C may proceed via a two stage process, wherein the first stage involves condensation of the compounds of Formulae A and B to yield a corresponding hydrazone intermediate (which can be optionally isolated), and subsequently transforming said hydrazone intermediate, via a rearrangement, to yield the compound of formula C (or formula I directly).

Suitably one or more of the following features apply in relation to the first stage of the reaction between the compounds of formulae A and B:

The reaction takes place in a suitable solvent, such as a protic solvent (suitably an alcoholic solvent such as ethanol).

The reaction takes place in the presence of a suitable acid, suitably a carboxylic acid, such as acetic acid.

The reaction takes place at a suitable temperature. A suitable temperature may be, for example, a temperature above 50° C., suitably above 70° C., or more suitably above 80° C.

The reaction takes place over a suitable time period (such as between 10 minutes and 5 hours, or between 0.5 and 2 hours).

Suitably one or more of the following features apply in relation to the second stage of the reaction between the compounds of formulae A and B (i.e. transformation of the hydrazone intermediate):

The reaction optionally takes place in a suitable solvent, such as a protic solvent (suitably an alcoholic solvent such as ethanol).

The reaction takes place in the presence of a suitable acid, suitably a phosphoric acid acid, such as polyphosphoric acid.

The reaction takes place at a suitable temperature. A suitable temperature may be, for example, a temperature above 80° C., suitably above 100° C., or more suitably above 110° C.

The reaction takes place over a suitable time period (such as between 10 minutes and 10 hours, or between 1 and 4 hours).

The hydrazone intermediate produced in stage one is suitably isolated from the reaction mixture. In a particular embodiment, the hydrazone is isolated by concentration of the reaction mixture to an oil (e.g. in vacuo). The oil is then suitably reacted directly in the second stage.

The product of the hydrazone transformation (i.e. the compound of Formula C) is suitably isolated by methods well known in the art. For instance, the compound of Formula C may be precipitated, suitably with cold water or ice, optionally following the addition of base (e.g. sodium hydroxide) to neutralise the mixture.

Scheme 2 depicts an alternative generalised scheme illustrating how the compounds of formula I may be synthesised:

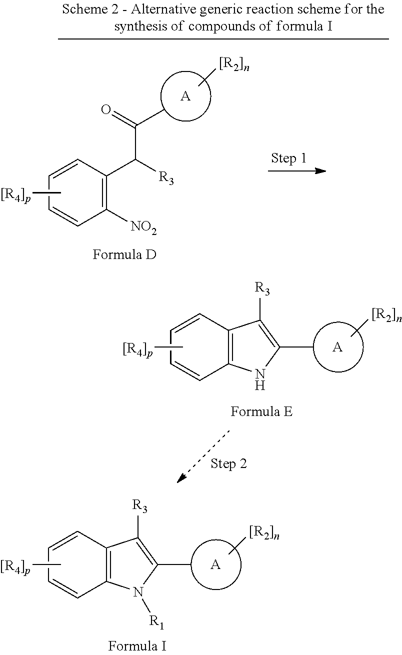

As before, R₁, R₂, R₃, R₄, n, p, Ring System A, and any other associated groups each have any one of the meanings defined herein. However, if R₁ is anything other than hydrogen, the R₁ group must be installed after Step 1.

The compounds of Formula D and E are intermediates from which a variety of compounds of formula I can be made.

Step 1 of Scheme 2 typically involves reduction of the nitro group, for instance with palladium on carbon (Pd/C) in an appropriate solvent (e.g. methanol). The reduced amino group may thereafter spontaneously cyclise via intramolecular reaction with the ketone group, to thereby yield the compound of Formula E (or formula I directly, for instance, where R₁ is hydrogen).

The compound of Formula D may be formed via a variety of routes known in the art. In a particular embodiment, where Ring A comprises a hydroxy group, especially a hydroxy group in a position ortho- to the carbon which ultimately becomes directly attached to the indole, the compound of Formula D may be formed via a Truce-Smiles rearrangement.^{[4]}

The present invention therefore further provides a process for preparing a compound of formula I (as defined herein), the process comprising the steps of:

reducing a compound of formula A:

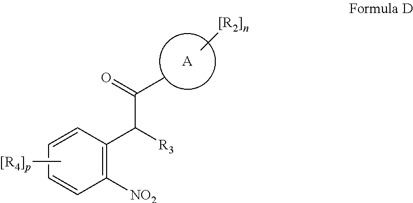

to form a compound of formula E:

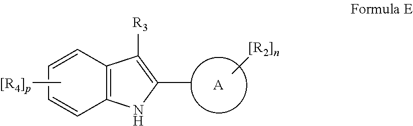

wherein R₂, R₃, R₄, n, p, Ring System A, and any other associated groups each have any one of the meanings defined herein;
and optionally thereafter (and if necessary—i.e. where R₁ in formula I is not hydrogen):
(i) Transforming the compound of Formula E into the compound of Formula I by, for example, installing an R₁ group upon the free indole nitrogen (i.e. where R₁ is other than hydrogen);
(ii) removing any protecting groups present;
(iii) converting the compound formula I into another compound of formula I;
(iv) forming a pharmaceutically acceptable salt, hydrate or solvate thereof; and/or forming a prodrug thereof.

Suitably one or more of the following features apply in relation to the conversion of the compound of Formula D to the compound of Formula E:

The reaction optionally takes place in a suitable solvent, such as a protic solvent (suitably an alcoholic solvent such as methanol or ethanol).

The reaction takes place at a suitable temperature (e.g. room temperature).

The reaction takes place over a suitable time period (such as between 1 hour and 24 hours).

The product of the nitro reduction reaction (i.e. the compound of Formula E) is suitably isolated by methods well known in the art. For instance, the compound of Formula E may be concentrated in vacuo and optionally further purified, for instance, via column chromatography or recrystallisation. In a further aspect of the invention, there is provided a compound of formula I obtainable by a process as defined herein.

In a further aspect of the invention, there is provided a compound of formula I obtained by a process as defined herein.

In a further aspect of the invention, there is provided a compound of formula I directly obtained by a process as defined herein.

In a further aspect of the present invention there is provided a novel intermediate compound of formula A, B, or C as defined herein.

### Biological Activity

The biological assay (i.e. the MTS assay) described in Example 8 herein may be used to measure the pharmacological effects of the compounds of the present invention.

Although the pharmacological properties of the compounds of the formulae I vary with structural change, as expected, the compounds of the invention were found to be active in the assay described in Example 8.

In general, the compounds of the invention demonstrate an IC₅₀ of 600 μM or less in the MTS assay with 1321N1 cell lines described in Example 8. Preferred compounds of the invention demonstrate an IC₅₀ of 500 μM or less in the MTS assay with 1321N1 cell lines described in Example 8. Most preferred compounds of the invention demonstrate an IC₅₀ of 450 μM or less in the MTS assay with 1321N1 cell lines described in Example 8. In a particular embodiment, the compounds of the invention demonstrate an IC₅₀ of 150 μM or less in the MTS assay with 1321N1 cell lines described in Example 8.

In general, the compounds of the invention demonstrate an IC₅₀ of 600 μM or less in the MTS assay with U87MG cell lines described in Example 8. Preferred compounds of the invention demonstrate an IC₅₀ of 500 μM or less in the MTS assay with U87MG cell lines described in Example 8. Most preferred compounds of the invention demonstrate an IC₅₀ of 450 μM or less in the MTS assay with U87MG cell lines described in Example 8. In a particular embodiment, the compounds of the invention demonstrate an IC₅₀ of 200 μM or less in the MTS assay with U87MG cell lines described in Example 8.

Suitably, the compounds of the invention demonstrate an IC₅₀ of 600 μM or less in the MTS assay with each of the 1321N1 and U87MG cell lines described in Example 8. Preferred compounds of the invention demonstrate an IC₅₀ of 500 μM or less in the MTS assay with each of the 1321N1 and U87MG cell lines described in Example 8. Most preferred compounds of the invention demonstrate an IC₅₀ of 450 μM or less in the MTS assay with each of the 1321N1 and U87MG cell lines described in Example 8. In a particular embodiment, the compounds of the invention demonstrate an IC₅₀ of 200 μM or less in the MTS assay with each of the 1321N1 and U87MG cell lines described in Example 8.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides a method of inhibiting growth of either or both U87MG and 1321N1 cell lines in vitro or in vivo, said method comprising contacting a cell with an effective amount of a compound, or a pharmaceutically acceptable salt, hydrate or solvate thereof, as defined herein.

The present invention provides a method of inhibiting cell proliferation, in vitro or in vivo, said method comprising contacting a cell with an effective amount of a a compound, or a pharmaceutically acceptable salt, hydrate or solvate thereof, as defined herein.

The present invention provides a method of treating a proliferative disorder in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of treating cancer in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use in the treatment of a proliferative condition.

The present invention provides a compound, or a pharmaceutically acceptable salt, hydrate or solvate thereof, or a pharmaceutical composition as defined herein for use in the treatment of cancer. In a particular embodiment, the cancer is human cancer.

The present invention, there is provided a compound, or a pharmaceutically acceptable salt, hydrate or solvate thereof, as defined herein for use in the inhibition of growth of either or both U87MG and 1321N1 cell lines.

The present invention provides a use of a compound, or a pharmaceutically acceptable salt, hydrate or solvate thereof, as defined herein in the manufacture of a medicament for the treatment of a proliferative condition.

The present invention provides a use of a compound, or a pharmaceutically acceptable salt, hydrate or solvate thereof, as defined herein in the manufacture of a medicament for the treatment of cancer. Suitably, the medicament is for use in the treatment of human cancers.

The present invention provides a use of a compound, or a pharmaceutically acceptable salt, hydrate or solvate thereof, as defined herein in the manufacture of a medicament for use in the inhibition of growth of either or both U87MG and 1321N1 cell lines.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compounds of the present invention have particular application in the treatment of human cancers, suitably by virtue of their inhibition of growth of either or both U87MG and 1321N1 cell lines.

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

In a particular embodiment of the invention, the proliferative condition to be treated is brain cancer and/or brain tumours. In an embodiment of the invention, the brain cancer is glioma.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy, wherein the chemotherapy may include one or more anti-tumour agents selected from procarbazine, carmustine, lomustine, irinotecan, temozolomide, cisplatin, carboplatin, methotrexate, etoposide, cyclophosphamide, ifosfamide, and vincristine.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and another anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### EXAMPLES

### General Experimental Details

Commercially available reagents were used as received without purification. Analytical thin layer chromatography (TLC) were performed with plastic-backed TLC plates coated with silica G/UV₂₅₄, in a variety of solvents. The plates were visualised by UV light (254 nm). Flash column chromatography was conducted with Davisil silica 60 Å (40-63 μm) under bellows pressure. Low resolution mass spectra were recorded on a Thermo Finnigan LCQ Advantage MAX using chemical ionisation (CI). ¹H and ¹³C NMR spectra were recorded on a Bruker Avance DPX 250 (250 MHz) or a Bruker 400 (400 MHz) spectrometer. All chemical shifts (6) are quoted in parts per million (ppm) relative to a calibration reference of the residual protic solvent; CHCl₃ (δ_{H} 7.26, s) or DMSO (δ_{H} 2.53, m) was used as the internal standard in ¹H NMR spectra, and ¹³C NMR shifts were referenced using CDCl₃ (δ_{C} 77.0, t) or DMSO (δ_{C} 40.5, sept) with broad band decoupling.

### Synthetic Scheme

The general synthetic scheme is shown above in Scheme 1.

Various general methods were used for the preparation of the examples described herein. The following procedures describe representative examples. From the foregoing and the disclosure that follows, the skilled person will readily understand how these syntheses can be adapted to derive the full range of compounds of the invention. Furthermore, the skilled person is readily able to overcome any synthetic challenges, without undue burden, by reference to appropriate literature which may be found by means of simple structure and/or substructure searches on online databases, such as SciFinder™ and Beilstein™

### Example 1

### Preparation of (1H-indol-3-yl)methanol (Compound 1)

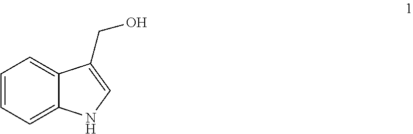 [SMILES: OCc1c[nH]c2ccccc12]

(1H-indol-3-yl)methanol was commercially obtained from Sigma-Aldrich and used, for comparative purposes, without further purification in the relevant assays.

### Example 2

### Preparation of 2-phenyl-1H-indole (Compound 2)

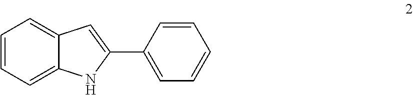 [SMILES: c1ccc(-c2cc3ccccc3[nH]2)cc1]

2-phenyl-1H-indole was commercially obtained from Sigma-Aldrich and used, for comparative purposes, without further purification in the relevant assays.

### Example 3

### Preparation of (2-phenyl-1H-indol-3-yl)methanol (Compound 3)^{[3]}

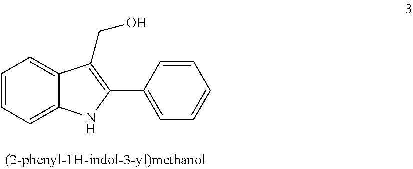 [SMILES: OCc1c(-c2ccccc2)[nH]c2ccccc12]

2-Phenylindole-3-carboxaldehyde (300 mg, 1.36 mmol) and NaBH₄ (103 mg, 2.72 mmol) were stirred at reflux in ethanol (10 mL) for 1 minute followed by stirring at room temperature for 2 hours. 1% NaOH (10 mL) was added to the reaction mixture and the product was extracted with Et₂O (3×10 mL). The combined extracts were dried (MgSO₄), filtered and the solvent evaporated to yield a crude product which was re-crystallised in PhMe:EtOAc:petroleum ether, yielding the title compound as a white solid 133 mg, 44%). ¹H NMR (250 MHz, DMSO-d6) δ_{H} 4.81 (d, J=5.0 Hz, 2H), 5.10 (t, J=5.0 Hz, 1H), 7.14-7.28 (m, 2H), 7.48-7.53 (m, 2H), 7.64 (t, J=7.5 Hz, 2H), 7.80 (d, J=7.5 Hz, 1H), 7.92 (d, J=7.5 Hz, 2H), 11.47 (s, 1H); ¹³C NMR (62.5 MHz, DMSO-d6), δ_{C} 54.8, 112.1, 113.3, 119.9, 122.6. 128.5, 128.9, 129.67, 129.71, 133.4, 136.8, 136.9; vₘₐₓ (film, cm⁻¹) 3488, 3183 (br.), 1638, 1491, 1452, 1392; m/z (CI) 206 ([M-OH]⁺, 100%).

### Example 4

### Preparation of 2-(1H-indol-2-yl)phenol (Compound 4)^{[4]}

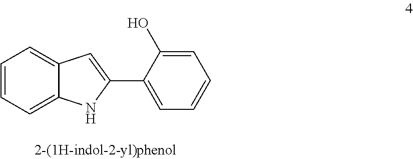 [SMILES: Oc1ccccc1-c1cc2ccccc2[nH]1]

1-(2-Hydroxyphenyl)-2-(2-nitrophenyl)ethanone,^{[4]} (105 mg, 0.41 mmol) was dissolved in methanol (4.1 mL). Pd/C (10 mg, 10 wt. %) was added and the flask evacuated and backfilled with hydrogen (3 cycles). The reaction was then stirred under an atmosphere of hydrogen for 18 h. The reaction was filtered through Celite® and eluted with methanol (10 mL) and the solvent removed in vacuo. The crude product was purified by column chromatography on silica gel (10% EtOAc in petroleum ether) to give the title compound as a pale yellow solid (62 mg, 72%). Rf 0.39 (30% ethyl acetate in petroleum ether). m.p. (EtOAc: petroleum ether) 170-172° C.; vₘₐₓ/cm⁻¹ 3500, 3425; δ_{H} (300 MHz; CDCl₃) 9.22 (1H, br. s), 7.70 (1H. dd. J=1.6 and 7.8 Hz, Ar), 7.66 (1H, d, J=7.8 Hz, Ar), 7.42 (1H, d, J=8.1 Hz, Ar), 7.26-7.11 (3H, m, Ar), 7.04 (1H, td, J=1.1 and 7.6 Hz, Ar), 6.91 (1H, dd, J=0.9 and 8.1 Hz, Ar), 6.87 (1H, m, Ar), 6.0-5.0 (1H, br. s); δ_{C} (75 MHz; CDCl₃) 152.0, 136.4, 134.8, 128.9, 128.4, 128.3, 122.2, 121.5, 120.4, 120.1, 119.1, 116.6, 111.0, 100.2; m/z (ES+) 210 ([M+H]⁺, 100%). Found 210.0920, C₁₄H₁₂NO (M+H⁺) requires 210.0919.

### Example 5

### Preparation of 2-phenyl-1H-indole-3-carbaldehyde (Compound 6)

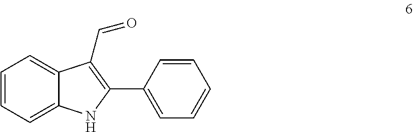 [SMILES: O=Cc1c(-c2ccccc2)[nH]c2ccccc12]

2-phenyl-1H-indole-3-carbaldehyde was commercially obtained from Sigma-Aldrich and used, for comparative purposes, without further purification in the relevant assays.

### Example 6

### Preparation of 2-(2-methoxyphenyl)-1H-indole (Compound 7)^{[5]}

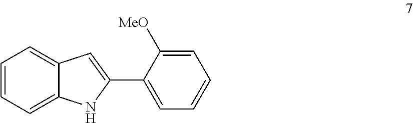 [SMILES: COc1ccccc1-c1cc2ccccc2[nH]1]

2′-Methoxyacetophenone (1.38 mL, 10 mmol) was mixed with phenylhydrazine (0.99 mL, 10 mmol) in ethanol (5 mL) and 4 drops of glacial acetic acid added. The pale yellow solution was heated to 80° C. with stirring for 1 hour which produced a red/brown solution. The solvent was evaporated to yield the phenylhydrazone intermediate as red/brown oil. To this oil was added polyphosphoric acid (20 g) and the reaction heated to 120° C. with stirring for 2 hours. After completion of the reaction (TLC) the reaction mixture was poured onto crushed ice, followed by the addition of NaOH until a neutral pH was reached. The product was extracted with DCM (3×50 mL) and the combined extracts washed with water (50 mL), brine (50 mL), dried (MgSO₄), filtered and the solvent evaporated. The product was purified using flash chromatography (SiO₂; 50% toluene: 50% petroleum ether) to yield the title compound as an off-white solid (532 mg, 24%). Rf 0.64 (100% toluene).

¹H NMR (250 MHz, CDCl₃) δ_{H} 4.03 (s, 3H), 6.93 (br. s, 1H) 7.03-7.34 (m, 5H), 7.46 (d, J=8.0 Hz, 1H), 7.67 (dd, J=0.5 and 7.5 Hz, 1H), 7.87 (dd, J=1.5 and 8.0 Hz, 1H), 9.70 (br. s, 1H); ¹³C NMR (62.5 MHz, CDCl₃) δ_{C} 55.8, 99.5, 110.9, 111.8, 119.8, 120.2, 120.5, 121.5, 121.8, 128.0, 128.3, 128.6, 135.9, 136.1, 155.7; vₘₐₓ (film, cm⁻¹) 3443, 1577, 1463, 1435, 1308, 1232; m/z (CI) 224 ([M+H]⁺, 100%).

### Example 7

### Preparation of cisplatin

Cisplatin was commercially obtained from Sigma-Aldrich and used, for comparative purposes, without further purification in the relevant assays.

### Example 8

### MTS Assay (Biological Activity)

The biological activity of the compounds of the invention can be assessed using an MTS assay as described further below.

MTS assay

1321N1 cells were maintained in Dulbeccos's Modified Eagle's Medium (DMEM) supplemented with 10% (v/v) foetal bovine serum (FBS) and 2 mM L-glutamine, while the U87MG cell line was maintained in Essential Minimum Eagle Medium (EMEM) supplemented with 10% (v/v) FBS, 2 mM L-glutamine, 1 mM sodium pyruvate and 1% (v/v) non-essential amino acids (NEAA) (Lonza, UK) in a 37° C. humidified incubator supplied with 5% CO₂. The 1321N1 and the U87MG cells were trypsinized and cultured overnight at a cell density of 3500 cells/200 μl and 4000 cells/200 μl respectively in 96-well microtitre plates (Sarstedt, UK). The following day when the cells looked 50-60% confluent, each concentration of the commercial drugs and novel compounds mentioned above were added in quadruplicates and left for incubation for 48 hours. After 48 hours, 20 μl of the pre-warmed MTS reagent (Promega, UK) was added to each of the wells and the 96-well plates were incubated at 37° C. humidified incubator supplied with 5% CO₂ for 60 min. At the end of 60 min, the absorbance was recorded at 490 nm using a Tecan GENios Pro® microplate reader. The concentration of compound that inhibits 50% of cell growth (IC₅₀) compared to untreated vehicle controls containing ≦0.5% absolute ethanol was calculated by nonlinear regression analysis. Absolute ethanol was used in the vehicle controls as indole 4 was dissolved in absolute ethanol.

Results

Initially indole compounds 1 and 2 were investigated to assess what kind of activity these compounds had against the established glioma cell lines 1321N1 (WHO Grade 2) and U87MG (WHO Grade 4). The results (Table 1, entries 1 and 2) reveal that whilst 13C (1) has modest activity (IC₅₀=374±2 μM (1321N1) and 585±2 μM (U87MG)), the 2-arylindole (2) does not reach its IC₅₀ over the same time frame (48 h) and only reaches a maximum growth inhibition of 7% (U87MG) at 400 μM, however, 2 is not able to inhibit the growth of the 1321N1 cell line.

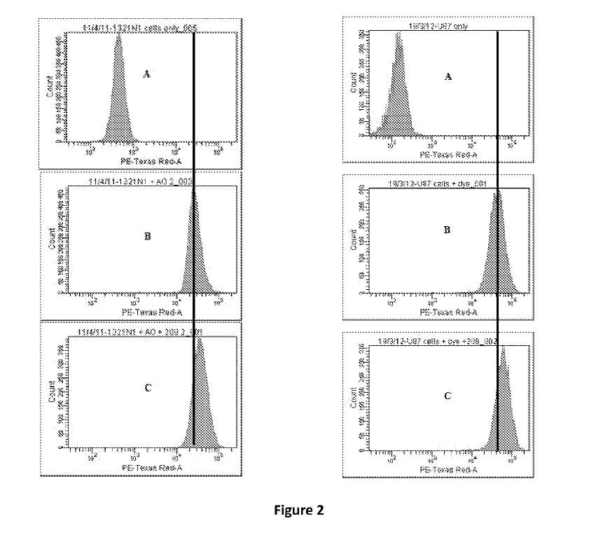

The inventors then sought to determine if making hybrid structures of 1 and 2, thus generating 3 and 4, would result in improved activity against these cell lines when compared to 1 and 2. In the event, indoles 3 and 4 were prepared as above and both were subjected to the same assay as indoles 1 and 2. The activities (Table 1, entries 3 and 4) reveal a surprising result; both hybrid compounds were more active than 2, in both cell lines, and both were more active than 1 in the malignant U87MG glioma cell line. Indoles 6 and 7 were also prepared or purchased, and evaluated in the same assay (Table 1, entries 5 and 6) as a test to determine the influence of the hydroxy group in these compounds. Interestingly, these two indoles (6 and 7) which lack the hydroxy group are both inactive, which in addition to the result with indole 2 (Table 1, entry 2), which also lacks this group, highlights the hydroxy's apparent importance for activity against these cell lines. All indoles assessed (FIG. 2 and Table 1) were screened alongside the known anticancer agent—cisplatin—to gain a measure of their therapeutic efficacy. Cisplatin was chosen because, in previous studies, it was shown to possess the best cytotoxic effects against these cell lines when compared to other known anticancer agents (temozolomide, etoposide and carmustine).^{[1]}

TABLE 1
A table to show the IC₅₀ values of the indoles against
two different cell lines using the MTS assay. The values shown
are the average and standard deviations of four repeats.

| | | MTS assay IC₅₀ values^{a} | |
|---|---|---|---|
| Entry | Compounds | 1321N1 (μM) | U87MG (μM) |
| 1 | 1(I3C) | 374 ± 2 | 585 ± 2 |
| 2 | 2^{b} | — | — |
| 3 | 3 | 111 ± 1 | 176 ± 1 |
| 4 | 4 | 445 ± 4 | 379 ± 4 |
| 5 | 6^{b} | — | — |
| 6 | 7^{b} | — | — |
| 7 | cisplatin | 11 ± 2 | 310 ± 4 |

^{a}IC₅₀ values reached after 48 h.
^{b}IC₅₀ value not reached.

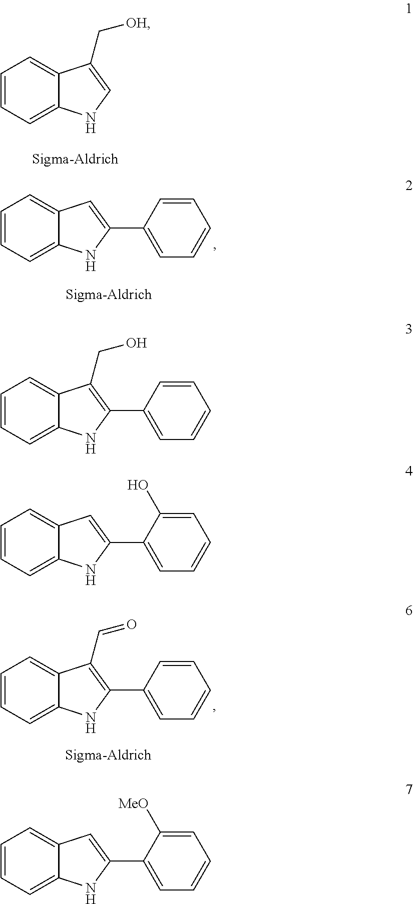 [SMILES: OCc1c[nH]c2ccccc12 ; c1ccc(-c2cc3ccccc3[nH]2)cc1 ; OCc1c(-c2ccccc2)[nH]c2ccccc12 ; Oc1ccccc1-c1cc2ccccc2[nH]1 ; O=Cc1c(-c2ccccc2)[nH]c2ccccc12 ; COc1ccccc1-c1cc2ccccc2[nH]1]

Only 1, 3, 4 and cisplatin were active against the 1321N1 cell line, with cisplatin showing an order of magnitude better activity than the indoles. However, it is of note that, against the U87MG cell line, compounds 3 and 4 had IC₅₀ values of a similar order of magnitude, if not better, than the established anticancer drug cisplatin. Evidently, the 2-Ph group is having a large influence on the activity of these indoles (compare entry 1 to entry 3 in Table 1).

The inventors then sought to determine the mechanism of action of the more active indoles (3 and 4).

Preliminary insights into their mechanism of action were gleaned from the structure of the active compounds. It was insightful that compounds 1, 3 and 4 were active, yet analogues such as 2, 6 and 7 were not; the hydroxy group was evidently having an effect. The inventors assumed that such a difference in activity could not be due to differences in hydrogen-bonding, and so the inventors turned their attention to the hydroxy or phenoxy groups in these compounds to investigate if they were responsible for the observed cell death.

### Example 9

### Reactive Oxygen Species Detection Assay

The inventors hypothesised that the active indoles were acting through a radical-based mechanism, which was most likely related to the generation of reactive oxygen species (ROS).^{[6]} As such, the inventors chose the Image-iT™ LIVE Green Reactive Oxygen Species Detection Kit from Invitrogen Ltd, UK, which is able to detect reactive oxygen species (ROS) in live cells, to assess selected indoles. Oxidatively stressed cells, when labeled with the carboxy-H₂DCFDA dye provided, should generate fluorescence which is detectable using a confocal microscope and flow cytometry. Various assays were therefore performed in accordance with the following protocols.

### Confocal

1321N1 cells were maintained in Dulbeccos's Modified Eagle's Medium (DMEM) supplemented with 10% (v/v) foetal bovine serum (FBS) and 2 mM L-glutamine, while the U87MG cell line was maintained in Essential Minimum Eagle Medium (EMEM) supplemented with 10% (v/v) FBS, 2 mM L-glutamine, 1 mM sodium pyruvate and 1% (v/v) non-essential amino acids (NEAA) (Lonza, UK) in a 37° C. humidified incubator supplied with 5% CO₂. The 1321N1 and the U87MG cells were trypsinized and cultured overnight at a cell density of 10⁴ cells/well/ml and were seeded in 6 well plates (BD Biosciences, UK) and were allowed to attach overnight. 2 ml of this cell suspension was added into each well resulting into a final concentration of 20,000 cells/well. On the day of experiment, cells were labelled with sufficient amount of 25 μM carboxy-H₂DCFDA dye and were incubated for 30 min at 37° C., protected from light. Cells were then treated with 500 μM of indole 4 and the positive control [tert-butyl hydroperoxide (TBHP)](100 μM) for 60 min. Cells were gently washed with Hankis balanced salt solution (HBSS) warm buffer three times and were imaged immediately on the Zeiss LSM510 laser confocal microscope at an excitation/emission of 495/529 nm. The untreated vehicle controls contained ≦0.5% absolute ethanol and DMSO as indole 4 was dissolved in absolute ethanol while TBHP was dissolved in DMSO.

### Flow Cytometry

1321N1 cells were maintained in Dulbeccos's Modified Eagle's Medium (DMEM) supplemented with 10% (v/v) foetal bovine serum (FBS) and 2 mM L-glutamine, while the U87MG cell line was maintained in Essential Minimum Eagle Medium (EMEM) supplemented with 10% (v/v) FBS, 2 mM L-glutamine, 1 mM sodium pyruvate and 1% (v/v) non-essential amino acids (NEAA) (Lonza, UK) in a 37° C. humidified incubator supplied with 5% CO₂. The 1321N1 and the U87MG cells were trypsinized and cultured overnight at a cell density of 10⁴ cells/well/ml and were seeded in 6 well plates (BD Biosciences, UK) and were allowed to attach overnight. 2 ml of this cell suspension was added into each well resulting into a final concentration of 20,000 cells/well. On the day of experiment, cells were labelled with sufficient amount of 25 μM carboxy-H₂DCFDA dye and were incubated for 30 min at 37° C., protected from light. Cells were then treated with 500 μM of indole 4 and positive control [tert-butyl hydroperoxide (TBHP)](100 μM) and negative control (indole 2) (500 μM) for 60 mins. Cells were gently washed with Hank□s balanced salt solution (HBSS) warm buffer three times and then trypsinized, centrifuged and resuspended in 300 μl PBS. Cells were filtered through cell strainer caps (35 μm mesh) to obtain a single cell suspension and analysed using a FACSAria flow cytometer (BD Biosciences). The untreated vehicle controls contained ≦0.5% absolute ethanol and DMSO as indole 4 was dissolved in absolute ethanol while TBHP and indole 2 were dissolved in DMSO.

### Acridine Orange Assay

1321N1 cells were maintained in Dulbeccos's Modified Eagle's Medium (DMEM) supplemented with 10% (v/v) foetal bovine serum (FBS) and 2 mM L-glutamine, while the U87MG cell line was maintained in Essential Minimum Eagle Medium (EMEM) supplemented with 10% (v/v) FBS, 2 mM L-glutamine, 1 mM sodium pyruvate and 1% (v/v) non-essential amino acids (NEAA) (Lonza, UK) in a 37° C. humidified incubator supplied with 5% CO₂. The 1321N1 and the U87MG cells were trypsinized and cultured overnight at a cell density of 10⁴ cells/well/ml and were seeded in 6 well plates (BD Biosciences, UK) and were allowed to attach overnight. 2 ml of this cell suspension was added into each well resulting into a final concentration of 20,000 cells/well. After 24 h of incubation, cells were treated with 500 μM of indole 4 for 60 min. After 60 min incubation with the compound the cells were stained with 3 μM acridine orange and incubated for 15 min at 37° C., protected from light. Cells were gently washed with PBS, trypsinized, centrifuged and were resuspended in 300 μl PBS. Cells were filtered through cell strainer caps (35 μm mesh) to obtain a single cell suspension and analysed using a FACSAria flow cytometer (BD Biosciences). The untreated vehicle controls contained ≦0.5% absolute ethanol as indole 4 was dissolved in absolute ethanol.

### Results

In the event, to confirm and quantify the amount of fluorescence (due to ROS) produced, glioma cell lines 1321N1 and U87MG were seeded in separate 6 well plates and were left overnight to attach. On the day of the experiment the carboxy-H₂DCFDA dye was added to the cells which were incubated for 30 min in the dark. Indoles 2, 3, 4 and 7 (500 μM) were then added, in separate experiments, and left for 1 h incubation. Cells were thoroughly washed at each step to prevent background staining and they were imaged immediately after incubation.

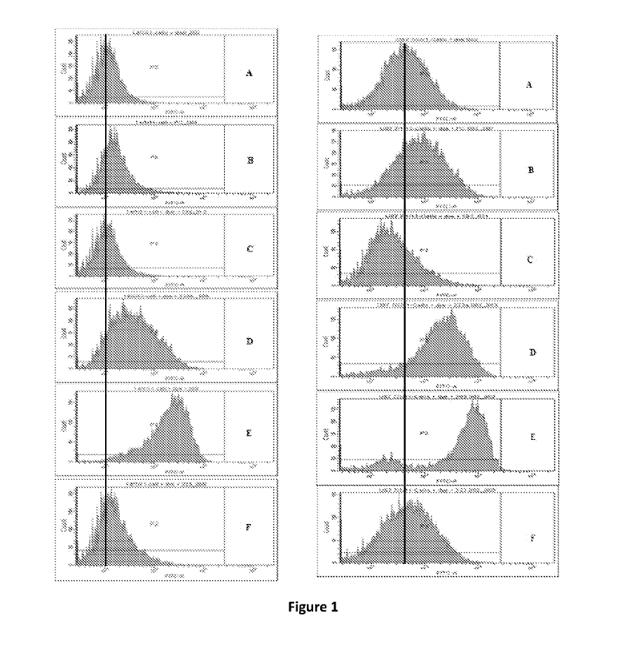

FIG. 1 shows H₂DCFDA staining intensity as quantified by flow cytometry. 1321N1 cells were treated with analogues of compound 4 for 1 h. The various charts of FIG. 1 show (A) Cells+Dye. (B) Cells treated with positive control [100 μM tert-butyl hydroperoxide] for 1 h. (C) Cells treated with indole 2 (500 μM) for 1 h. (D) Cells treated with indole 3 (500 μM) for 1 h. (E) Cells treated with indole 4 (500 μM) for 1 h. (F) Cells treated with indole 7 (500 μM) for 1 h. (Right) U87MG cells were treated with analogues of compound 4 for 1 h. (A) Cells+Dye. (B) Cells treated with positive control [100 μM tert-butyl hydroperoxide (TBHP)] for 1 h. (C) Cells treated with indole 2 (500 μM) for 1 h. (D) Cells treated with indole 3 (500 μM) for 1 h. (E) Cells treated with indole 4 (500 μM) for 1 h. (F) Cells treated with indole 7 (500 μM) for 1 h.

The results (FIG. 1) suggest that the cells were under oxidative stress when in the presence of indoles 3 and 4 (FIG. 1, D and E), as identified by the shift of the fluorescence signal in these cases compared to the control signal (FIG. 1, A), but that oxidative stress was not observed with 2 and 7 (FIG. 1, C and F) in which no shift was observed compared to the control signal. Presumably, only 3 and 4 generated ROS as a result of the hydroxy group in their structures being able to form radical species in vitro upon reaction with redox enzymes. Indole 7 was used as a comparison (which has the hydroxy group masked as a methoxy group) and it was found that ROS was not induced in this case, as would be expected if the free hydroxy group was important for ROS generation. Nor was it induced by indole 2, which lacks any oxygenation on the 2-phenyl group.

The positive control used in this experiment was tert-butyl hydroperoxide (TBHP) (100 μM) which is a common inducer of ROS and was suggested and provided by Invitrogen Ltd. Importantly, the indoles inducing ROS (3 and 4) appeared to induce it to a greater level than the positive control (TBHP), suggesting that these compounds are powerful inducers of reactive oxygen species and that this presumably is intrinsic in their ability to affect the viability and hence proliferation of the cell lines tested. In particular, the results suggest that the fluorescence generated in the 1321N1 and U87MG cell lines treated with indole 4 were significantly higher when compared to the controls (FIG. 3), confirming that the cell death observed may be due to the formation of excessive ROS placing the cells under oxidative stress.

Ascorbic acid is a naturally occurring organic compound with antioxidant properties and is a scavenger of hydroxy radicals, reacting with reactive oxygen species, such as the hydroxy radical, and neutralizing it.^{[7]} Recent studies have found that ascorbic acid inhibits ROS production by neutralizing the radicals formed,^{[8]} and therefore ascorbic acid was chosen here to validate the ROS production induced by indole 4 (500 μM) in the 1321N1 and U87MG cell lines following the assumption that the inventors would see reduced amounts of ROS production after the co-treatment of the cells with ascorbic acid (100 μM) [8] and indole 4. At this stage the inventors decided to concentrate on the phenol derivative 4 since, in their hands, carbinol 3 had proved to be slightly unstable under acidic conditions.

The results of the ascorbic acid treatment reveals that the level of fluorescence produced by the 1321N1 cells treated with a combination of indole 4 and ascorbic acid was less than the vehicle control cells alone (data not shown), thus further validating that indole 4 expresses its cytotoxic behaviour through ROS generation, but that this cannot happen in the presence appropriate levels of radical scavengers.^{[9]} This was also supported by the MTS assay on both cell lines, which showed that cell viability was unaffected by indole 4, but only when in the presence of ascorbic acid.

To further understand the effects that such ROS generation was having on the cells the inventors examined whether indole 4 was responsible for cell death through the induction of autophagy, as has been observed for other small molecules.^{[10], [11], [12], [13], [14], [15], [16]}

Oxidative stress is known to induce autophagy,^{[11][13][15][16][17]} and cell death associated with autophagy in tumour cell lines treated with chemotherapeutic agents has been proposed recently.^{[18]} Such research has also shown that cell death is associated with autophagy in malignant gliomas,^{[10]} ovarian carcinomas^{[19]} and mammary carcinomas,^{[20]} and that hydrogen peroxide (H₂O₂) and 2-methoxyestradiol (2-ME) induce oxidative stress, causing autophagic cell death, in the transformed cell line HEK293 and cancer cell lines U87MG and HeLa cells.^{[17]} Furthermore, β-lapachone induces ROS generation which mediates autophagic cell death in U87MG cells.^{[}10] The phenolic compound, resveratrol, has also been shown to induce autophagy in human U251MG glioma cells.^{[21]}

Based on such precedent the inventors set out to see if autophagy was being induced in the 1321N1 and U87MG cells upon treatment with the most active, stable compound, indole 4. The process of autophagy is in-part characterized by the formation of acidic vesicular organelles (AVOs) which can be detected and measured by staining with acridine orange. Acridine orange moves freely across biological membranes and accumulates in acidic compartments upon protonation where it can be observed and quantified by fluorescence. Therefore, in view of the finding that AVOs accumulate in cells undergoing autophagy, the inventors examined whether indole 4 induced AVO formation.

The glioma cells were seeded in 6 well plates and were left overnight to attach. The cells were then treated with indole 4 for 1 h, stained with acridine orange for 15 min, and were analysed by flow cytometry.

FIG. 2 shows acridine orange staining intensity, as measured by flow cytometry, of acidic vesicular organelle (AVO) formation in 1321N1 cells (Left) and U87MG cells (Right). The various charts of FIG. 2 show (A) Cells only. (B) Cells+acridine orange. (C) Cells treated with indole 4 (500 μM) for 1 h+acridine orange.

The results (FIG. 2) indicated the formation of AVOs had occurred, as concluded by the increased amount of red fluorescence in cell lines studied. This result suggests that indole 4 induces AVO formation as a result of autophagic cell death which is mediated by reactive oxygen species generation in these particular cell lines, and potentially reveals a new class of compound which could possibly be optimized further for glioma treatment through analogue preparation.

Based on these preliminary results the inventors were keen to discover if the effects would also be observed against a primary cell culture (IN859) from cells obtained from a biopsy from a glioblastoma multiforme patient. The IC₅₀ value obtained (398±3 μM) is comparable to that for U87MG (379±4 μM) suggesting that indole 4 has the ability to kill primary tissue as well as established cancer cell lines.

### REFERENCES

[1]S. Prabhu, F. Harris, R. Lea and T. J. Snape, Neuro-Oncology, 2011, 13, 12-12.
[2] Shou, J; Ali-Osman, F; Multani, A S; Pathak, S; Fedi, P; Srivenugopal, K S, Oncogene, 2002, 21, 6, p 878-889.
[3]E. Leete, J. Am. Chem. Soc., 1959, 81, 6023-6026.
[4]T. J. Snape, Synlett, 2008, 2689-2691.
[5]C. M. So, C. P. Lau and F. Y. Kwong, Org. Lett., 2007, 9, 2795-2798.
[6] Y.-M. Han, D.-S. Shin, Y.-J. Lee, I. A. Ismail, S.-H. Hong, D. C. Han and B.-M. Kwon, Bioorg Med Chem Lett, 2011, 21, 747-751.
[7] S. Biswas, J. Bhattacharyya and A. G. Dutta, Mol. Cell. Biochem., 2005, 276, 205-210.
[8] Y. Peng, K. H. H. Kwok, P. H. Yang, S. S. M. Ng, J. Liu, O. G. Wong, M. L. He, H. F. Kung and M. C. M. Lin, Neuropharmacology, 2005, 48, 426-434.
[9]C.-W. Tsai, C.-Y. Lin, H.-H. Lin and J.-H. Chen, Neurochemical Research, 2011, 36, 2442-2451.
[10]E. J. Parka, K. S. Choi and T. K. Kwon, Chemico-Biological Interactions, 2011, 189, 37-44.
[11]W.-J. Duan, Q.-S. Li, M.-Y. Xia, S.-I. Tashiro, S. Onodera and T. Ikejima, Journal of Asian Natural Products Research, 2011, 13, 27-35.
[12]R. A. Gonzalez-Polo, M. Niso-Santano, M. A. Ortiz-Ortiz, A. Gomez-Martin, J. M.

Moran, L. Garcia-Rubio, J. Francisco-Morcillo, C. Zaragoza, G. Soler and J. M. Fuentes, Autophagy, 2007, 3, 366-367.
[13]J. Li, X.-L. Wang, Y.-C. Fang and C.-Y. Wang, Journal of Asian Natural Products Research, 2010, 12, 992-1000.
[14]J.-y. Wu, K.-w. Tsai, J.-j. Shee, Y.-z. Li, C.-h. Chen, J.-j. Chuang and Y.-w. Liu, Acta Pharmacologica Sinica, 2010, 31, 81-92.
[15]D. Xiao, A. A. Powolny, J. Antosiewicz, E.-R. Hahm, A. Bommareddy, Y. Zeng, D. Desai, S. Amin, A. Herman-Antosiewicz and S. V. Singh, Pharmaceutical Research, 2009, 26, 1729-1738.
[16]36. Y.-h. Zhang, Y.-I. Wu, S.-i. Tashiro, S. Onodera and T. Ikejima, Acta Pharmacologica Sinica, 2011, 32, 1266-1275.
[17] Y. Chen, E. McMillan-Ward, J. Kong, S. J. Israels and S. B. Gibson, Cell Death and Differentiation, 2008, 15, 171-182.
[18] B. Levine and J. Y. Yuan, Journal of Clinical Investigation, 2005, 115, 2679-2688.
[19]A. W. Opipari, L. J. Tan, A. E. Boitano, D. R. Sorenson, A. Aurora and J. R. Liu, Cancer Research, 2004, 64, 696-703.
[20]W. Bursch, A. Ellinger, H. Kienzl, L. Torok, S. Pandey, M. Sikorska, R. Walker and R. S. Hermann, Carcinogenesis, 1996, 17, 1595-1607.
[21]J. Li, Z. H. Qin and Z. Q. Liang, BMC Cancer, 2009, 9.

1. A method for treating a proliferative disorder, comprising administering to a patient in need thereof a compound defined by Formula Ib: 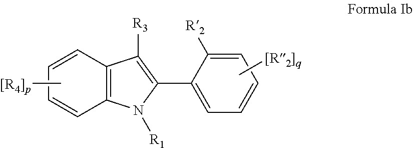
wherein:
R₁ is selected from the group including hydrogen, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulphamoyl, N,N-di-[(1-6C)alkyl]sulphamoyl, or from a group of the formula:
-L^{1a}-X^{1a}
wherein:
L^{1a} is absent or is selected from SO, SO₂, CO, C(O)O, CH(OR₁ₐ), CON(R₁ₐ), SO₂N(R₁ₐ), wherein R₁ₐ is hydrogen or (1-8C)alkyl; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₁ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:
-L^{1b}-X^{1b}
wherein:
L^{1b} is absent or is selected from O, S, SO, SO₂, N(R_{1b}), CO, C(O)O, CH(OR_{1b}), CON(R_{1b}), N(R_{1b})CO, N(R₁ₐ)CON(R_{1b}), SO₂N(R_{1b}), N(R_{1b})SO₂, OC(R_{1b})₂, SC(R_{1b})₂ and N(R_{1b})C(R_{1b})₂, wherein R_{1b} is hydrogen or (1-8C)alkyl; and
X^{1b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
R′₂ is hydrogen or is selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{2a}-X^{2a}
wherein:
L^{2a} is absent or is selected from O, S, SO, SO₂, N(R₂ₐ), CO, C(O)O, CH(OR₂ₐ), CON(R₂ₐ), N(R₂ₐ)CO, N(R₂ₐ)CON(R₂ₐ), SO₂N(R₂ₐ), N(R₂ₐ)SO₂, OC(R₂ₐ)₂, SC(R₂ₐ)₂ and N(R₂ₐ) C(R₂ₐ)₂, wherein R₂ₐ is hydrogen or (1-8C)alkyl; and
X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R′₂ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{2b}-X^{2b}
wherein:
L^{2b} is absent or is selected from O, S, SO, SO₂, N(R_{2b}), CO, C(O) O, CH(OR_{2b}), CON(R_{2b}), N(R_{2b})CO, N(R₂ₐ)CON(R_{2b}), SO₂N(R_{2b}), N(R_{2b})SO₂, OC(R_{2b})₂, SC(R_{2b})₂ and N(R_{2b}) C(R_{2b})₂, wherein R_{2b} is hydrogen or (1-8C)alkyl; and
X^{2b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
q is 0, 1, 2, or 3;
each R″₂ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{2a}-X^{2a};
wherein each R″₂ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{2b}-X^{2b};
R₃ is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{3a}-X^{3a}
wherein:
L^{3a} is absent or is selected from O, S, SO, SO₂, N(R₃ₐ), CO, C(O)O, CH(OR₃ₐ), CON(R₃ₐ), N(R₃ₐ) CO, N(R₃ₐ)CON(R₃ₐ), SO₂N(R₃ₐ), N(R₃ₐ)SO₂, OC(R₃ₐ)₂, SC(R₃ₐ)₂ and N(R₃ₐ)C(R₃ₐ)₂, wherein R₃ₐ is hydrogen or (1-8C)alkyl; and
X^{3a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₃ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:
-L^{3b}-X^{3b}
wherein:
L^{3b} is absent or is selected from O, S, SO, SO₂, N(R_{3b}), CO, C(O) O, CH(OR_{3b}), CON(R_{3b}), N(R_{3b})CO, N(R₃ₐ)CON(R_{3b}), SO₂N(R_{3b}), N(R_{3b})SO₂, OC(R_{3b})₂, SC(R_{3b})₂ and N(R_{3b}) C(R_{3b})₂, wherein R_{3b} is hydrogen or (1-8C)alkyl; and
X^{3b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
p is 0, 1, 2, 3, or 4;
each R₄ group, which may be the same or different, is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
-L^{4a}-X^{4a}
wherein:
L^{4a} is absent or is selected from O, S, SO, SO₂, N(R₄ₐ), CO, C(O)O, CH(OR₄ₐ), CON(R₄ₐ), N(R₄ₐ)CO, N(R₄ₐ)CON(R₄ₐ), SO₂N(R₄ₐ), N(R₄ₐ)SO₂, OC(R₄ₐ)₂, SC(R₄ₐ)₂ and N(R₄ₐ) C(R₄ₐ)₂, wherein R₄ₐ is hydrogen or (1-8C)alkyl; and
X^{4a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein R₄ is optionally further substituted with one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, 2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-C)alkanesulphonylamino and N-(1-6C)alkyl-(1-6C)alkanesulphonylamino, or from a group of the formula:
-L^{4b}-X^{4b}
wherein:
L^{4b} is absent or is selected from O, S, SO, SO₂, N(R_{4b}), CO, C(O) O, CH(OR_{4b}), CON(R_{4b}), N(R_{4b})CO, N(R₄ₐ)CON(R_{4b}), SO₂N(R_{4b}), N(R_{4b})SO₂, OC(R_{4b})₂, SC(R_{4b})₂ and N(R_{4b})C(R_{4b})₂, wherein R_{4b} is hydrogen or (1-8C)alkyl; and
X^{4b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein at least one of the R′₂ group, the R₃ group, or an R₄ group in the 4-indole position is or comprises a hydroxy, amino, NH(Rₓ), or mercapto group, wherein Rₓ is any acceptable group, defined in relation to R′₂, R₃, and R₄, for attachment to a nitrogen atom;
or a pharmaceutically acceptable salt, hydrate or solvate thereof.

2. The method of claim 1, wherein at least one of the R′₂ group, the R₃ group, or an R₄ group in the 4-indole position, is or comprises a hydroxyl.

3. The method of claim 2, wherein at least one of R′₂ and the R₃ group is or comprises a hydroxyl.

4. The method of claim 1, wherein q is 0, and the compound has the structural formula Id:

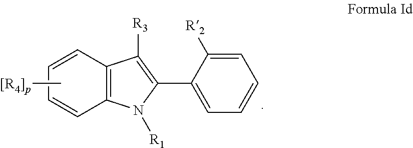

5. The method of claim 1, wherein p is 0.

6. The method of claim 1, wherein R₁ is hydrogen.

7. The method of claim 1, wherein one of R′₂ or R₃ is or comprises a hydroxy, amino, NH(Rₓ), or mercapto group, wherein Rₓ is any acceptable group, defined herein in relation to R₂ and R₃, for attachment to a nitrogen atom, and the other of R′₂ or R₃ is hydrogen.

8. The method of claim 7, wherein one of R′₂ or R₃ is or comprises a hydroxy, and the other of R′₂ or R₃ is hydrogen.

9. The method of claim 1, wherein R′₂ is or comprises a group selected from hydroxy, amino, NH(Rₓ), or mercapto, wherein Rₓ is any acceptable group, defined in relation to R′₂, for attachment to a nitrogen atom.

10. The method of claim 1, wherein R′₂ is hydroxy.

11. The method of claim 1, wherein R₃ is hydrogen or is a group selected from hydroxy, amino, NH(Rₓ), or mercapto, wherein Rₓ is any acceptable group, defined herein in relation to R₃, for attachment to a nitrogen atom.

12. The method of claim 1, wherein R₃ is selected from hydrogen, hydroxy, (1-4C)hydroxyalkyl.

13. The method of claim 1, wherein one of R′₂ or R₃ is hydroxy or hydroxymethyl, and the other of R′₂ or R₃ is hydrogen.

14. The method of claim 1, wherein at least one R₄ group is or comprises a group selected from hydroxy, amino, NH(Rₓ), or mercapto, wherein Rₓ is any acceptable group, defined in relation to R₄, for attachment to a nitrogen atom.

15. The method of claim 1, wherein the compound is selected from:
(2-phenyl-1H-indol-3-yl)methanol;
2-(1H-indol-2-yl)phenol;
or a pharmaceutically acceptable salt, hydrate or solvate thereof.

16. The method of claim 1, wherein the proliferative disorder is cancer.

17-20. (canceled)

21. The method of claim 16, wherein the cancer is brain cancer.

22. The method of claim 21, wherein the brain cancer is a glioma.

23. The method of claim 16, wherein the treating further comprises administering another anti-tumor agent to the patient in combination with the compound of formula I(b).

24. A method of inhibiting cell proliferation, in vitro or in vivo, comprising contacting an abnormal cell that undergoes unwanted or uncontrolled proliferation with an effective amount of a compound of formula Ib.
